(19) United States
(12) Patent Application Publication
(10) Pub. No.: US 2021/0301320 A1
(43) Pub. Date: Sep. 30, 2021

(54) MULTIPLEXED OPTIMIZED MISMATCH AMPLIFICATION (MOMA)-CANCER RISK ASSESSMENT WITH NON-CANCER ASSOCIATED TARGETS

(71) Applicant: The Medical College of Wisconsin, Inc., Milwaukee, WI (US)

(72) Inventors: Aoy Tomita Mitchell, Elm Grove, WI (US); Karl Stamm, Brookfield, WI (US)

(73) Assignee: The Medical College of Wisconsin, Inc., Milwaukee, WI (US)

(21) Appl. No.: 17/093,298

(22) Filed: Nov. 9, 2020

Related U.S. Application Data
(63) Continuation of application No. PCT/US2019/031860, filed on May 10, 2019.
(60) Provisional application No. 62/669,950, filed on May 10, 2018.

(51) Int. Cl. C12Q 1/686; C12Q 1/6806

(52) U.S. Cl. CPC C12Q 1/686; C12Q 1/6806

(57) ABSTRACT

This invention relates to methods and compositions for assessing an amount of non-native nucleic acids in a sample, such as from a subject, and/or noise, background or discordance quality check (QC). The methods and compositions provided herein can be used to determine risk of a condition, such as cancer, in a subject.

### RELATED APPLICATION

This application claims the benefit of priority under 35 U.S.C. § 119(c) of the filing date of U.S. Provisional Application No. 62/669,950, filed May 10, 2018, the entire contents of which is incorporated by reference herein.

### FIELD OF THE INVENTION

This invention relates to methods and compositions for assessing an amount of non-native nucleic acids in a sample from a subject and/or the background, noise or discordance quality check (QC). The methods and compositions provided herein can be used to determine risk of a condition, such as cancer. In some embodiments of any one of the methods provided herein the background, noise or discordance QC correlates with cancer risk. Accordingly, in any one of the methods provided herein the background, noise or discordance QC can be used to assess cancer risk in a subject. This invention further relates to methods and compositions for assessing the amount of non-native cell-free deoxyribonucleic acid (non-native cell-free DNA) and/or the background, noise or discordance QC, such as using multiplexed optimized mismatch amplification (MOMA), in some aspects.

### SUMMARY OF INVENTION

The present disclosure is based, at least in part on the surprising discovery that background, noise or discordance quality check (QC) can be used to identify subjects with or at risk of cancer by quantifying low frequency non-native nucleic acids in a sample from a subject and/or background, noise or discordance QC. In some embodiments, the nucleic acids can be measured using multiplexed optimized mismatch amplification (MOMA). In some embodiments, the nucleic acids can be measured using a sequencing-based method. In any one of the methods provided herein the steps of a MOMA assay can be replaced with steps of a sequencing-based method, such as comprising any one or more steps of a sequencing-based as provided herein or otherwise known in the art.

Multiplexed optimized mismatch amplification embraces the design of primers that can include a 3′ penultimate mismatch for the amplification of a specific sequence but a double mismatch relative to an alternate sequence. Amplification with such primers can permit the quantitative determination of amounts of non-native nucleic acids in a sample and/or background, noise or discordance QC.

The methods, compositions or kits can be any one of the methods, compositions or kits, respectively, provided herein, including any one of those of the examples and drawings.

Surprisingly, when analyzing the level of non-native nucleic acids in a sample from a subject from a foreign source, such as another subject, the level of non-native nucleic acids (and/or noise or background or discordance QC in performing the assay) was found to be indicative of cancer risk. Thus, any one of the methods, compositions or kits provided herein are related to using MOMA to obtain such values and assessing risk of cancer. Preferably, the assay is performed with a “universal” panel of targets (i.e., a panel of targets designed based on SNV information within a population and without regard to the genotype of the native or non-native nucleic acids or specific mutations related to a disease or condition), in some embodiments.

The disclosure, in some aspects, provides a method of assessing an amount of non-native nucleic acids in a sample from a subject having, at risk of, suspected of having, or previously had cancer and/or noise or background or discordance QC, the sample comprising non-native and native nucleic acids, the method comprising: for a plurality single nucleotide variant (SNV) targets, performing an amplification-based quantification assay on the sample, or portion thereof, with at least two primer pairs, wherein each primer pair comprises a forward primer and a reverse primer, wherein one of the at least two primer pairs comprises a 3′ penultimate mismatch in a primer relative to one allele of the SNV target but a 3′ double mismatch relative to another allele of the SNV target and specifically amplifies the one allele of the SNV target, and the another of the at least two primer pairs specifically amplifies the another allele of the SNV target, and wherein at least one of the SNV targets is not a cancer-specific SNV target, and obtaining or providing results from the amplification-based quantification assays to determine the amount of non-native nucleic acids and/or the background, noise or discordance QC in the sample.

In one embodiment of any one of the methods provided, none of the SNV targets are cancer-specific SNV targets. In one embodiment of any of the methods provided, the results are provided in a report. In one embodiment of any of the methods provided, the method further comprises determining the amount of the non-native nucleic acids and/or noise or background or discordance QC in the sample based on the results. In one embodiment of any of the methods provided, the results comprise the amount of the non-native nucleic acids and/or noise or background or discordance QC in the sample. In one embodiment of any of the methods provided, the method further comprises determining the level of background, noise or discordance QC based on the results.

In one aspect, a method of assessing an amount of non-native nucleic acids in a sample from a subject having, at risk of, suspected of having, or previously had cancer and/or noise or background or discordance QC, the sample comprising non-native and native nucleic acids, the method comprising: obtaining results from an amplification-based quantification assay performed on the sample, or portion thereof, wherein the assay comprises amplification of a plurality of single nucleotide variant (SNV) targets with at least two primer pairs for each of the SNV targets, wherein each primer pair comprises a forward primer and a reverse primer, wherein one of the at least two primer pairs comprises a 3′ penultimate mismatch in a primer relative to one allele of the SNV target but a 3′ double mismatch relative to another allele of the SNV target and specifically amplifies the one allele of the SNV target, and another of the at least two primer pairs specifically amplifies the another allele of the SNV target, and wherein at least one of the SNV targets is not a cancer-specific SNV informative target, and assessing the amount of non-native nucleic acids and/or noise, background or discordance QC based on the results.

In one embodiment of any of the methods provided herein, the amount of the non-native nucleic acids in the sample and/or noise, background or discordance QC is based on the results of the amplification-based quantification assays. In one embodiment of any one of the methods provided herein, the results are obtained from a report.

In one embodiment of any of the methods provided herein, the another primer pair of the at least two primer pairs also comprises a 3′ penultimate mismatch relative to the another allele of the SNV target but a 3′ double mismatch relative to the one allele of the SNV target in a primer and specifically amplifies the another allele of the SNV target.

In one embodiment of any of the methods provided herein, the amount is an absolute value for the non-native nucleic acids and/or noise or background or discordance QC in the sample. In one embodiment of any one of the methods provided herein, the amount is a relative value for the non-native nucleic acids and/or noise or background or discordance QC in the sample. In one embodiment of any one of the methods provided herein, the amount is the ratio or percentage of non-native nucleic acids to native nucleic acids or total nucleic acids.

In one embodiment of any of the methods provided herein, the method further comprises obtaining the genotype of the non-native nucleic acids and/or native nucleic acids. In one embodiment of any one of the methods provided herein, the method further comprises obtaining the at least two primer pairs for each of the SNV targets.

In one embodiment of any of the methods provided herein, the amount of non-native nucleic acids in the sample is at least 5%. In one embodiment of any one of the methods provided herein, the amount of non-native nucleic acids in the sample is at least 10%. In one embodiment of any one of the methods provided herein, the amount of non-native nucleic acids in the sample is at least 15%. In one embodiment of any one of the methods provided herein, the amount of non-native nucleic acids in the sample is at least 20%.

In one embodiment of any of the methods provided herein, the sample comprises cell-free DNA and the amount is an amount of non-native cell-free DNA and/or noise or background or discordance QC.

In one embodiment of any of the methods provided herein, the subject is a transplant recipient. In one embodiment of any one of the methods provided herein, the subject has, is at risk of having, is suspected of having, or previously had a hematological cancer. In one embodiment of any one of the methods provided herein, the hematological cancer is lymphoma.

In one embodiment of any of the methods provided herein, the amplification-based quantification assays are quantitative PCR assays, such as real time PCR assays or digital PCR assays.

In one embodiment of any of the methods provided herein, the method further comprises determining a risk associated with cancer in the subject based on the amount of non-native nucleic acids and/or noise, background or discordance QC in the sample. In one embodiment of any one of the methods provided herein, the risk is increased if the amount of non-native nucleic acids and/or noise, background or discordance QC is greater than a threshold value. In one embodiment of any one of the methods provided herein, the risk is decreased if the amount of non-native nucleic acids and/or noise, background or discordance QC is less than a threshold value.

In one embodiment of any of the methods provided herein, the method further comprises selecting a treatment for the subject based on the amount of non-native nucleic acids and/or noise, background or discordance QC in the sample. In one embodiment of any one of the methods provided herein, the treatment is a cancer treatment.

In one embodiment of any of the methods provided herein, the method further comprises treating the subject based on the amount of non-native nucleic acids and/or noise, background or discordance QC in the sample.

In one embodiment of any of the methods provided herein, the method further comprises providing information about a treatment to the subject, or suggesting non-treatment, based on the amount of non-native nucleic acids and/or noise, background or discordance QC in the sample.

In one embodiment of any of the methods provided herein, the method further comprises monitoring or suggesting the monitoring of the amount of non-native nucleic acids and/or noise, background or discordance QC in the subject over time. In one embodiment of any of the methods provided herein, the method further comprises assessing the amount of non-native nucleic acids and/or noise, background or discordance QC in the subject at a subsequent point in time.

In one embodiment of any of the methods provided herein, the method further comprises evaluating an effect of a treatment administered to the subject based on the amount of non-native nucleic acids and/or noise, background or discordance QC.

In one embodiment of any of the methods provided herein, the method further comprises providing or obtaining the sample or a portion thereof.

In one embodiment of any of the methods provided herein, the method further comprises extracting nucleic acids from the sample.

In one embodiment of any one of the methods provided herein, the method further comprises a pre-amplification step using primers for the SNV targets. The primers may be the same or different as those for determining the amount of non-native nucleic acids.

In one embodiment of any one of the methods provided herein, the probe in one or more or all of the PCR quantification assays is on the same strand as the mismatch primer and not on the opposite strand.

In one embodiment of any one of the methods provided herein, the sample comprises blood, plasma or serum.

In one aspect, a method comprising obtaining the amount of non-native nucleic acids and/or noise, background or discordance QC based on any one of the methods provided herein is provided. In one embodiment, the method is the method of any one of the claims.

In one embodiment of any one of the methods provided herein, the method further comprises assessing a risk associated with cancer in the subject based on the amount of non-native nucleic acids and/or noise, background or discordance QC.

In one embodiment of any of the methods provided herein, the subject is a recipient of a transplant. In one embodiment of any of the methods provided herein, a treatment or information about a treatment or non-treatment is selected for or provided to the subject based on the assessed risk.

In one embodiment of any of the methods provided herein, the method further comprises monitoring or suggesting the monitoring of the amount of non-native nucleic acids and/or noise, background or discordance QC in the subject over time.

In one aspect, a report containing one or more of the results as provided herein is provided. In one embodiment of any one of the reports provided, the report is in electronic form. In one embodiment of any one of the reports provided, the report is a hard copy. In one embodiment of any one of the reports provided, the report is given orally.

In one embodiment of any one of the methods, compositions or kits provided, the mismatched primer(s) is/are the forward primer(s). In one embodiment of any one of the methods, compositions or kits provided, the reverse primers for the primer pairs for each SNV target is the same.

In one embodiment, any one of the embodiments for the methods provided herein can be an embodiment for any one of the compositions, kits or reports provided. In one embodiment, any one of the embodiments for the compositions, kits or reports provided herein can be an embodiment for any one of the methods provided herein.

### BRIEF DESCRIPTION OF DRAWINGS

The accompanying drawings are not intended to be drawn to scale. The figures are illustrative only and are not required for enablement of the disclosure.

FIG. 1 provides an exemplary, non-limiting diagram of MOMA primers. In a polymerase chain reaction (PCR) assay, extension of the sequence containing SNV A is expected to occur, resulting in the detection of SNV A, which may be subsequently quantified. Extension of the SNV B, however, is not expected to occur due to the double mismatch.

FIG. 2 demonstrates the use of expectation maximization to predict non-native (such as donor) genotype when unknown. Dashed line=first iteration, Solid line=second iteration, Final call=10%.

FIG. 3 demonstrates the use of expectation maximization to predict non-native (such as donor) genotype when unknown. Final call=5%.

FIG. 4 provides reconstruction experiment data demonstrating the ability to predict the non-native (such as donor) genotype when unknown. Data have been generated with a set of 95 SNV targets.

FIG. 5 provides an example of the average background noise for 104 MOMA targets.

FIG. 6 provides further examples of the background noise for methods using MOMA.

FIGS. 7 and 8 provide examples of an elevated donor fraction in two patient samples. The data is tabulated in Table 1.

FIG. 9 illustrates an example of a computer system with which some embodiments may operate.

### DETAILED DESCRIPTION OF THE INVENTION

Aspects of the disclosure relate to methods for the sensitive detection and/or quantification of non-native nucleic acids in a sample and/or noise, background or discordance quality check (QC). Non-native nucleic acids, such as non-native DNA, may be present in individuals, for example, individuals with cancer. The disclosure provides techniques to detect, analyze and/or quantify non-native nucleic acids, such as non-native cell-free DNA concentrations, and/or noise, background or discordance QC in samples obtained from a subject.

In one embodiment, in any one of the methods provided herein the level of nucleic acids, such as non-native nucleic acids, may be determined by a MOMA assay. In one embodiment, any one of the methods provided herein the level of such nucleic acids may be determined with any one of the methods of PCT Publication No. WO 2016/176662 A1, and such methods are incorporated herein by reference in their entirety.

In another embodiment, in any one of the methods provided herein the level of nucleic acids, such as non-native nucleic acids, may be determined with any one of the methods of U.S. Application No. 62/547,098, and such methods are incorporated herein by reference in their entirety.

In another embodiment, in any one of the methods provided herein the level of nucleic acids, such as non-native nucleic acids, may be determined with a sequencing-based method, and, accordingly, the steps for performing a MOMA assay in any one of the methods provided herein are replaced with steps for performing a sequencing-based method. The sequencing-based methods is as provided herein or otherwise known in the art. For example, the nucleic acids, such as non-native nucleic acids, may be measured by analyzing the DNA of a sample to identify multiple loci, an allele of each of the loci may be determined, and informative loci may be selected based on the determined alleles. As used herein, “loci” refer to nucleotide positions in a nucleic acid, e.g., a nucleotide position on a chromosome or in a gene. As used herein, “informative loci” refers to a locus where the genotype of the subject is homozygous for the major allele, while the genotype of the donor is homozygous or heterozygous for the minor allele. As used herein, “minor allele” refers to the allele that is less frequent in the population of nucleic acids for a locus. In some embodiments, the minor allele is the nucleotide identity at the locus in the nucleic acid of the donor. A “major allele”, on the other hand, refers to the more frequent allele in a population. In some embodiments, the major allele is the nucleotide identity at the locus in the nucleic acid of the subject.

In some embodiments, the informative loci and alleles can be determined based on prior genotyping, such as of the nucleic acids of the subject and the nucleic acids of the donor. For example, the genotype of the recipient and donor can be compared, and informative loci can be identified as those loci where the recipient is homozygous for a nucleotide identity and the donor is heterozygous or homozygous for a different nucleotide identity. Methods for genotyping are well known in the art and further described herein. In this example, the minor and major allele may be identified by determining the relative quantities of each allele at the informative locus and/or may be identified as the nucleotide identity at the informative locus in the donor DNA (minor allele) and the recipient DNA (major allele). Accordingly, the methods provided can further include a step of genotyping, such as of the recipient and donor, or obtaining or being provided with such genotypes.

An estimated allele frequency, such as the estimated minor allele frequency, at the informative loci may then be calculated in a suitable manner. In some embodiments, the estimated allele frequency may be calculated based on modeling the number of counts of the allele, such as the minor allele, at the informative loci using a statistical distribution. For example, the estimated allele frequency can be calculated by modeling allele read counts using a binomial distribution. In some embodiments, the peak of such a distribution is determined and is indicative of the percent donor-specific cf-DNA. A frequency of the minor allele at the informative loci may also be calculated using a maximum likelihood method. In some embodiments, the minor allele frequency (MAF) may be calculated, such as with genotypes from plasma DNA of the subject, and donor genotypes for informative loci may be inferred using expectation maximization. In some embodiments, the read counts for the major and/or minor allele(s) can be corrected prior to estimating the allele frequency.

The DNA may be analyzed using any suitable next generation or high-throughput sequencing and/or genotyping technique. Examples of next generation and high-throughput sequencing and/or genotyping techniques include, but are not limited to, massively parallel signature sequencing, polony sequencing, 454 pyrosequencing, Illumina (Solexa) sequencing, SOLiD sequencing, ion semiconductor sequencing, DNA nanoball sequencing, heliscope single molecule sequencing, single molecule real time (SMRT) sequencing, MassARRAY®, and Digital Analysis of Selected Regions (DANSR™) (see, e.g., Stein RA (1 Sep. 2008). “Next-Generation Sequencing Update”. Genetic Engineering & Biotechnology News 28 (15); Quail, Michael; Smith, Miriam E; Coupland. Paul; Otto, Thomas D; Harris, Simon R; Connor, Thomas R; Bertoni, Anna; Swerdlow, Harold P; Gu, Yong (1 Jan. 2012). “A tale of three next generation sequencing platforms: comparison of Ion torrent, pacific biosciences and illumina MiSeq sequencers”. BMC Genomics 13 (1): 341; Liu, Lin; Li, Yinhu; Li, Siliang; Hu, Ni; He, Yimin; Pong, Ray; Lin, Danni; Lu, Lihua; Law, Maggie (1 Jan. 2012). “Comparison of Next-Generation Sequencing Systems”. Journal of Biomedicine and Biotechnology 2012: 1-11; Qualitative and quantitative genotyping using single base primer extension coupled with matrix-assisted laser desorption/ionization time-of-flight mass spectrometry (MassARRAY®). Methods Mol Biol. 2009; 578:307-43; Chu T, Bunce K, Hogge W A, Peters D G. A novel approach toward the challenge of accurately quantifying fetal DNA in maternal plasma. Prenat Diagn 2010; 30:1226-9; and Suzuki N, Kamataki A, Yamaki J, Homma Y. Characterization of circulating DNA in healthy human plasma. Clinica chimica acta; International Journal of Clinical Chemistry 2008; 387:55-8).

In one embodiment, any one of the methods for determining the level of nucleic acids, such as non-native nucleic acids, may be any one of the methods of U.S. Publication No. 2015-0086477-A1, and such methods are incorporated herein by reference in their entirety. Again, such a method may replace the steps of performing a MOMA assay in any one of the methods provided herein.

As used herein, “non-native nucleic acids” refers to nucleic acids that are from another source or are mutated versions of a nucleic acid found in a subject (with respect to a specific sequence, such as a wild-type (WT) sequence). “Native nucleic acids”, therefore, are nucleic acids that are not from another source and are not mutated versions of a nucleic acid found in a subject (with respect to a specific sequence). In some embodiments, the non-native nucleic acid is non-native cell-free DNA. “Cell-free DNA” (or cf-DNA) is DNA that is present outside of a cell, e.g., in the blood, plasma, serum, urine, etc. of a subject. Without wishing to be bound by any particular theory or mechanism, it is believed that cf-DNA is released from cells, e.g., via apoptosis of the cells. An example of non-native nucleic acids are nucleic acids that are from a cancer in a subject. As used herein, the compositions and methods provided herein can be used to determine an amount of cell-free DNA from a non-native source and/or noise, background or discordance QC related to the determination.

Provided herein are methods and compositions that can be used to measure nucleic acids with differences in sequence identity. In some embodiments, the difference in sequence identity is a single nucleotide variant (SNV); however, wherever a SNV is referred to herein any difference in sequence identity between native and non-native nucleic acids is intended to also be applicable. Thus, any one of the methods or compositions provided herein may be applied to native versus non-native nucleic acids where there is a difference in sequence identity. As used herein, “single nucleotide variant” refers to a nucleic acid sequence within which there is sequence variability at a single nucleotide. In some embodiments, the SNV is a biallelic SNV, meaning that there is one major allele and one minor allele for the SNV. In some embodiments, the SNV may have more than two alleles, such as within a population. In some embodiments, the SNV is a mutant version of a sequence, and the non-native nucleic acid refers to the mutant version, while the native nucleic acid refers to the non-mutated version (such as the wild-type version). Such SNVs, thus, can be mutations that can occur within a subject and which can be associated with a disease or condition. Generally, a “minor allele” refers to an allele that is less frequent, such as in a population, for a locus, while a “major allele” refers to the more frequent allele, such as in a population. The methods and compositions provided herein can quantify nucleic acids of major and minor alleles within a mixture of nucleic acids even when present at low levels and/or noise, background or discordance QC related to such quantification, in some embodiments.

In some embodiments of any one of the methods provided herein, the SNV targets may include mutations specific to or that can identify a cancer, referred to as “cancer-specific SNVs”. In some preferred embodiments, however, some or all of the SNV targets are not associated with cancer.

The nucleic acid sequence within which there is sequence identity variability, such as a SNV, is generally referred to as a “target”. As used herein, a “SNV target” refers to a nucleic acid sequence within which there is sequence variability, such as at a single nucleotide, such as in a population of individuals or as a result of a mutation that can occur in a subject and that can be associated with a disease or condition. The SNV target has more than one allele, and in preferred embodiments, the SNV target is biallelic. In some embodiments of any one of the methods provided herein, the SNV target is a single nucleotide polymorphism (SNP) target. In some of these embodiments, the SNP target is biallelic. It has been discovered that non-native nucleic acids can be quantified even at extremely low levels by performing amplification-based quantitative assays, such as PCR assays with primers specific for SNV targets. From such quantitative assays the related noise, background or discordance QC may be determined in addition to or instead of as an indicator of risk as provided herein. In some embodiments, the amount of non-native nucleic acids and/or noise, background or discordance QC is determined by performing amplification-based quantitative assays, such as quantitative PCR assays, with primers for a plurality of SNV targets.

As used herein, “discordance quality check” or “discordance quality control” (“discordance QC” or “dQC”) refers to a measurement of background noise resulting from a mismatch between the expected genotype (e.g., the native and non-native genotypes) and the experimental genotype. The metric is used for quality assurance purposes, and is usually computed as a safeguard against sample mixups and contamination. It represents the median value reported at loci believed to be non-informative (i.e., the same genotype is homozygous in the native and non-native nucleic acid samples). Similar to the background noise of an assay, it is elevated in cases of genomic instability or incorrect subject-sample assignment.

dQC can be measured using any method known in the art. For example, when calculating non-native fraction, the presence of native and non-native genotyping information is assumed. The data is examined to find genomic targets which are homozygous in both the native and non-native genotypes and have the same allele (e.g., non-informative loci). The genomic targets, since they are the same in both the native and non-native genotypes are therefore indistinguishable. Any measured minor species then corresponds to the background noise (dQC) of that genomic target.

In some instances, the discordance QC may have minor species measurement ranging from 0.1% to 100%, for example, greater than 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 65%, 70%, 75%, 80%, 85%, 90%, 95%, 96%, 97%, 98%, or 99%. In some embodiments, the dQC may be 100%. Without wishing to be bound by theory, it is thought that higher levels of dQC (e.g., levels above 50%) may indicate that the incorrect genotyping information was used during the analysis. Likewise, more subtle elevations, for example, between 0.1% and 10%, may indicate sample contamination.

A “plurality of SNV targets” refers to more than one SNV target where for each target there are at least two alleles. Preferably, in some embodiments, each SNV target is expected to be biallelic and a primer pair specific to each allele of the SNV target is used to specifically amplify nucleic acids of each allele, where amplification occurs if the nucleic acid of the specific allele is present in the sample. In some embodiments, the plurality of SNV targets are a plurality of sequences within a subject that can be mutated and that if so mutated can be indicative of a disease or condition in the subject. In some embodiments of any one of the methods provided herein, an amplification-based quantification assay, such as quantitative PCR, is performed with primer pairs for at least 6 informative targets. In some embodiments of any one of the methods provided herein, an amplification-based quantification assay, such as quantitative PCR, is performed with primer pairs for at least 6 but less than 35 informative targets, less than 30 informative targets, less than 25 informative targets, less than 20 informative targets, less than 15 informative targets, or less than 10 informative targets.

In some embodiments of any one of the methods or compositions provided herein, informative results are obtained with primer pairs for at least 18, 21, 24, 27, 30, 33, 36, 39, 42, 45, 48, 51, 54, 57, 60, 63, 66, 69, 72, 75, 78, 81, 84, 87 or more targets. In some embodiments of any one of the methods or compositions provided herein, the quantitative assay is performed with primer pairs for fewer than 96, 93, 90, 87, 84, 81, 78, 75, 72 or 69 targets. In some embodiments of any one of the methods or compositions provided herein, the quantitative assay is performed with primer pairs for 18-30, 18-45, 18-60, 18-75, 18-80, 18-85, 18-90 or 18-95 targets. In some embodiments of any one of the methods or compositions provided herein, the quantitative assay is performed with primer pairs for 21-30, 21-45, 21-60, 21-75, 21-80, 21-85, 21-90 or 21-95 targets. In some embodiments of any one of the methods or compositions provided herein, the quantitative assay is performed with primer pairs for 24-30, 24-45, 24-60, 24-75, 24-80, 24-85, 24-90 or 24-95 targets. In some embodiments of any one of the methods or compositions provided herein, the quantitative assay is performed with primer pairs for 30-45, 30-60, 30-75, 30-80, 30-85, 30-90 or 30-95 targets. In some embodiments of any one of the methods or compositions provided herein, the quantitative assay is performed with primer pairs for 40-45, 40-60, 40-75, 40-80, 40-85, 40-90 or 40-95 targets. In some embodiments of any one of the methods or compositions provided herein, the quantitative assay is performed with primer pairs for 45-60, 45-75, 45-80, 45-85, 45-90 or 45-95 targets. In some embodiments of any one of the methods or compositions provided herein, the quantitative assay is performed with primer pairs for 50-60, 50-75, 50-80, 50-85, 50-90 or 50-95 targets. For any one of the methods or compositions provided, the method or composition can be directed to any one of the foregoing numbers of targets or informative targets.

In some embodiments of any one of the methods provided herein, at least one of the primer pairs is not for a cancer-specific SNV target. In another embodiment of any one of the methods provided herein, one or more, including all, of the primer pairs are not for cancer-specific SNV targets. In an embodiment of any one of the methods or compositions provided herein, primer pairs for SNV targets can be pre-selected based on knowledge that the SNV targets will be informative, such as with knowledge of genotype. In another embodiment of any one of the methods or compositions provided herein, however, primer pairs for SNV targets are selected for the likelihood a percentage will be informative. In such embodiments, primer pairs for a greater number of SNV targets are used based on the probability a percentage of which will be informative.

In some embodiments, the subject may have previously had cancer and the method is for assessing the recurrence of the cancer. In some embodiments, the subject may have been previously diagnosed with cancer, and the method is for monitoring the cancer over time.

As used herein, “an informative SNV target” is one in which amplification with primers as provided herein occurs, and the results of which are informative. “Informative results” as provided herein are the results that can be used to quantify the level of non-native and/or native nucleic acids in a sample. In some embodiments, informative results exclude results where the native nucleic acids are heterozygous for a specific SNV target as well as “no call” or erroneous call results. From the informative results, allele percentages can be calculated using standard curves, in some embodiments of any one of the methods provided. In some embodiments of any one of the methods provided, the amount of non-native and/or native nucleic acids represents an average across informative results for the non-native and/or native nucleic acids, respectively.

The amount or level, such as ratio or percentage, of non-native nucleic acids may be determined with the quantities of the major and minor alleles as well as the genotype of the native and/or non-native nucleic acids. For example, results where the native nucleic acids are heterozygous for a specific SNV target may be excluded with knowledge of the native genotype. Further, results can also be assessed with knowledge of the non-native genotype. In some embodiments of any one of the methods provided herein, where the genotype of the native nucleic acids is known but the genotype of the non-native nucleic acids is not known, the method may include a step of predicting the likely non-native genotype or determining the non-native genotype by sequencing. Further details of such methods can be found, for example, in PCT Publication No. WO2016/176662, and such methods are incorporated by reference herein. In some embodiments of any one of the methods provided herein, the alleles can be determined based on prior genotyping of the native nucleic acids of the subject and/or the nucleic acids not native to the subject. Methods for genotyping are well known in the art. Such methods include sequencing, such as next generation, hybridization, microarray, other separation technologies or PCR assays. Any one of the methods provided herein can include steps of obtaining such genotypes.

“Obtaining” as used herein refers to any method by which the respective information or materials can be acquired. Thus, the respective information can be acquired by experimental methods, such as to determine the native genotype. Respective materials can be created, designed, etc. with various experimental or laboratory methods, in some embodiments. The respective information or materials can also be acquired by being given or provided with the information, such as in a report, or materials. Materials may be given or provided through commercial means (i.e. by purchasing), in some embodiments.

Reports may be in oral, written (or hard copy) or electronic form, such as in a form that can be visualized or displayed. In some embodiments, the “raw” results for each assay as provided herein are provided in a report, and from this report, further steps can be taken to determine the amount of non-native nucleic acids and/or noise, background or discordance QC in the sample. These further steps may include any one or more of the following, selecting informative results, obtaining the native and/or non-native genotype, calculating allele percentages for informative results for the native and non-native nucleic acids, averaging the allele percentages, etc.

In other embodiments, the report provides the amount of non-native nucleic acids and/or noise, background or discordance QC in the sample. From the amount, in some embodiments, a clinician may assess the need for a treatment for the subject or the need to monitor the amount of the non-native nucleic acids and/or noise, background or discordance QC over time. Accordingly, in any one of the methods provided herein, the method can include assessing the amount of non-nucleic acids and/or noise, background or discordance QC at more than one point in time. In some embodiments, the amount of non-nucleic acids and/or noise, background or discordance QC is assessed prior to, during, or following a treatment. In some embodiments, the amount of non-nucleic acids and/or noise, background or discordance QC is used to determine the success of a treatment. For example, the amount of non-nucleic acids and/or noise, background or discordance QC of a subject may be monitored every 1, 2, 3, 4, 5, 6, or 7 days; 2, 3, 4, 5, 6, 7, or 8 weeks; monthly; annually, or less frequently. If the amount of non-nucleic acids and/or noise, background or discordance QC drops below a certain threshold (e.g., 0.5%, 1.0%, 1.5%, or more or any one of the thresholds provided herein), less frequent monitoring may be indicated. Conversely, if the amount of non-nucleic acids and/or noise, background or discordance QC rises above a certain threshold (e.g., 0.5%, 1.0%, 1.5%, or more or any one of the thresholds provided herein), more frequent monitoring may be indicated. Levels above a certain threshold may also be indicative that alternative or additional treatment is needed. Such decisions are within the purview of the medical professional. Such assessing can be performed with any one of the methods or compositions provided herein.

In some embodiments, any one of the methods provided herein may include a step of determining or obtaining the total amount of nucleic acids, such as total cell-free DNA, in one or more samples from the subject. Accordingly, any one or more of the reports provided herein may also include one or more amounts of the total nucleic acids, such as total cell-free DNA, and it is the combination of the amount of non-native nucleic acids and/or noise, background or discordance QC and total nucleic acids that is in a report and from which a clinician may assess the need for a treatment for the subject or the need to monitor the subject. As used herein, “total nucleic acids” refers to the total amount of nucleic acids in a sample. In some preferred embodiments of any one of the methods provided herein, the total amount of nucleic acids is determined by a MOMA assay as provided herein and is a measure of native and non-native nucleic acid counts as determined by the MOMA assay, preferably, from informative targets. In some embodiments, the total amount of nucleic acids is determined by any method such as a MOMA assay as provided herein or other assays known to those of ordinary skill in the art but not a MOMA assay as provided herein.

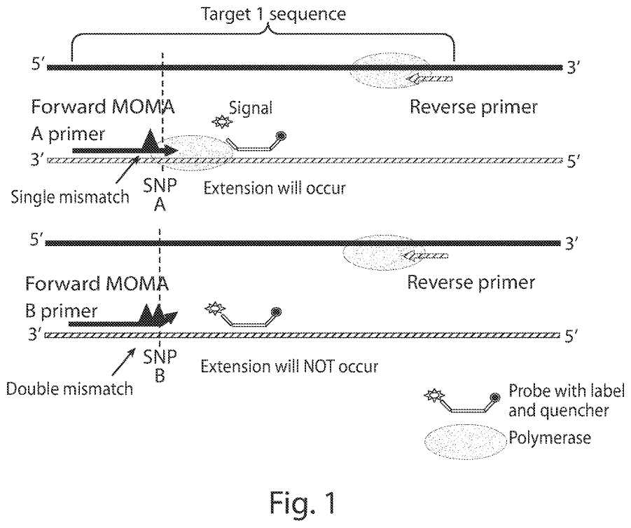

The quantitative assays as provided herein can make use of multiplexed optimized mismatch amplification (MOMA). Primers for use in such assays may be obtained, and any one of the methods provided herein can include a step of obtaining one or more primer pairs for performing the quantitative assays. Generally, the primers possess unique properties that facilitate their use in quantifying amounts of nucleic acids. For example, a forward primer of a primer pair can be mismatched at a 3′ nucleotide (e.g., penultimate 3′ nucleotide). In some embodiments of any one of the methods or compositions provided, this mismatch is at a 3′ nucleotide but adjacent to the SNV position. In some embodiments of any one of the methods or composition provided, the mismatch positioning of the primer relative to a SNV position is as shown in FIG. 1. Generally, such a forward primer, even with the 3′ mismatch, is able to produce an amplification product (in conjunction with a suitable reverse primer) in an amplification reaction, thus allowing for the amplification and resulting detection of a nucleic acid with the respective SNV. If the particular SNV is not present, and there is a double mismatch with respect to the other allele of the SNV target, an amplification product will generally not be produced. Preferably, in some embodiments of any one of the methods or compositions provided herein, for each SNV target a primer pair is obtained whereby specific amplification of each allele can occur without amplification of the other allele(s). “Specific amplification” refers to the amplification of a specific allele of a target without substantial amplification of another nucleic acid or without amplification of another nucleic acid sequence above background or noise. In some embodiments, specific amplification results only in the amplification of the specific allele.

In some embodiments of any one of the methods or compositions provided herein, for each SNV target that is biallelic, there are two primer pairs, each specific to one of the two alleles and thus have a single mismatch with respect to the allele it is to amplify and a double mismatch with respect to the allele it is not to amplify (if the nucleic acids of these alleles are present). In some embodiments of any one of the methods or compositions provided herein, the mismatch primer is the forward primer. In some embodiments of any one of the methods or compositions provided herein, the reverse primer of the two primer pairs for each SNV target is the same.

These concepts can be used in the design of primer pairs for any one of the compositions and methods provided herein. It should be appreciated that the forward and reverse primers are designed to bind opposite strands (e.g., a sense strand and an antisense strand) in order to amplify a fragment of a specific locus of the template. The forward and reverse primers of a primer pair may be designed to amplify a nucleic acid fragment of any suitable size to detect the presence of, for example, an allele of a SNV target, according to the disclosure. Any one of the methods provided herein can include one or more steps for obtaining one or more primer pairs as described herein.

It should be appreciated that primer pairs described herein may be used in a multiplex PCR assay. Accordingly, in some embodiments of any one of the methods or compositions provided herein, the primer pairs are designed to be compatible with other primer pairs in a PCR reaction. For example, the primer pairs may be designed to be compatible with at least 2, at least 5, at least 10, at least 15, at least 20, at least 25, at least 30, at least 35, at least 40, etc. other primer pairs in a PCR reaction. As used herein, primer pairs in a PCR reaction are “compatible” if they are capable of amplifying their target in the same PCR reaction. In some embodiments, primer pairs are compatible if the primer pairs are inhibited from amplifying their target DNA by no more than 1%, no more than 2%, no more than 3%, no more than 4%, no more than 5%, no more than 10%, no more than 15%, no more than 20%, no more than 25%, no more than 30%, no more than 35%, no more than 40%, no more than 45%, no more than 50%, or no more than 60% when multiplexed in the same PCR reaction. Primer pairs may not be compatible for a number of reasons including, but not limited to, the formation of primer dimers and binding to off-target sites on a template that may interfere with another primer pair. Accordingly, the primer pairs of the disclosure may be designed to prevent the formation of dimers with other primer pairs or to limit the number of off-target binding sites. Exemplary methods for designing primers for use in a multiplex PCR assay are known in the art or otherwise described herein.

In some embodiments, the primer pairs described herein are used in a multiplex PCR assay to quantify an amount of non-native nucleic acids and/or the noise, background or discordance QC of such an assay. Accordingly, in some embodiments of any one of the methods or compositions provided herein, the primer pairs are designed to detect genomic regions that are diploid, excluding primer pairs that are designed to detect genomic regions that are potentially non-diploid. In some embodiments of any one of the methods or compositions provided herein, the primer pairs used in accordance with the disclosure do not detect repeat-masked regions, known copy-number variable regions, or other genomic regions that may be non-diploid.

In some embodiments of any one of the methods provided herein, the amplification-based quantitative assay is any quantitative assay, such as whereby nucleic acids are amplified and the amounts of the nucleic acids can be determined. Such assays include those whereby nucleic acids are amplified with the MOMA primers as described herein and quantified. Such assays include simple amplification and detection, hybridization techniques, separation technologies, such as electrophoresis, next generation sequencing and the like.

In some embodiments of any one of the methods provided herein, the quantitative assays are quantitative PCR assays. Quantitative PCR include real-time PCR, digital PCR, TAQMAN™, etc. In some embodiments of any one of the methods provided herein the PCR is “real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau.

The use of multiple probes can expand the capability of single-probe real-time PCR. Multiplex real-time PCR uses multiple probe-based assays, in which each assay can have a specific probe labeled with a unique fluorescent dye, resulting in different observed colors for each assay. Real-time PCR instruments can discriminate between the fluorescence generated from different dyes. Different probes can be labeled with different dyes that each have unique emission spectra. Spectral signals are collected with discrete optics, passed through a series of filter sets, and collected by an array of detectors. Spectral overlap between dyes may be corrected by using pure dye spectra to deconvolute the experimental data by matrix algebra.

A probe may be useful for methods of the present disclosure, particularly for those methods that include a quantification step. Any one of the methods provided herein can include the use of a probe in the performance of the PCR assay(s), while any one of the compositions of kits provided herein can include one or more probes. Importantly, in some embodiments of any one of the methods provided herein, the probe in one or more or all of the PCR quantification assays is on the same strand as the mismatch primer and not on the opposite strand. It has been found that in so incorporating the probe in a PCR reaction, additional allele-specific discrimination can be provided.

As an example, a TAQMAN™ probe is a hydrolysis probe that has a FAM™ or VIC® dye label on the 5′ end, and minor groove binder (MGB) non-fluorescent quencher (NFQ) on the 3′ end. The TAQMAN™ probe principle generally relies on the 5′-3′ exonuclease activity of Taq® polymerase to cleave the dual-labeled TAQMAN™ probe during hybridization to a complementary probe-binding region and fluorophore-based detection. TAQMAN™ probes can increase the specificity of detection in quantitative measurements during the exponential stages of a quantitative PCR reaction.

PCR systems generally rely upon the detection and quantitation of fluorescent dyes or reporters, the signal of which increase in direct proportion to the amount of PCR product in a reaction. For example, in the simplest and most economical format, that reporter can be the double-strand DNA-specific dye SYBR® Green (Molecular Probes). SYBR Green is a dye that binds the minor groove of double stranded DNA. When SYBR Green dye binds to a double-stranded DNA, the fluorescence intensity increases. As more double-stranded amplicons are produced, the SYBR Green dye signal will increase.

In any one of the methods provided herein, the PCR may be digital PCR. Digital PCR involves partitioning of diluted amplification products into a plurality of discrete test sites such that most of the discrete test sites comprise either zero or one amplification product. The amplification products are then analyzed to provide a representation of the frequency of the selected genomic regions of interest in a sample. Analysis of one amplification product per discrete test site results in a binary “yes-or-no” result for each discrete test site, allowing the selected genomic regions of interest to be quantified and the relative frequency of the selected genomic regions of interest in relation to one another to be determined. In certain aspects, in addition to or as an alternative, multiple analyses may be performed using amplification products corresponding to genomic regions from predetermined regions. Results from the analysis of two or more predetermined regions can be used to quantify and determine the relative frequency of the number of amplification products. Using two or more predetermined regions to determine the frequency in a sample can reduce a possibility of bias through, e.g., variations in amplification efficiency, which may not be readily apparent through a single detection assay. Methods for quantifying DNA using digital PCR are known in the art and have been previously described, for example in U.S. Patent Publication number US20140242582, and such methods are incorporated herein by reference.

It should be appreciated that the PCR conditions provided herein may be modified or optimized to work in accordance with any one of the methods described herein. Typically, the PCR conditions are based on the enzyme used, the target template, and/or the primers. In some embodiments, one or more components of the PCR reaction is modified or optimized. Non-limiting examples of the components of a PCR reaction that may be optimized include the template DNA, the primers (e.g., forward primers and reverse primers), the deoxynucleotides (dNTPs), the polymerase, the magnesium concentration, the buffer, the probe (e.g., when performing real-time PCR), the buffer, and the reaction volume.

In any of the foregoing embodiments, any DNA polymerase (enzyme that catalyzes polymerization of DNA nucleotides into a DNA strand) may be utilized, including thermostable polymerases. Suitable polymerase enzymes will be known to those skilled in the art, and include E. coli DNA polymerase, Klenow fragment of E. coli DNA polymerase I, T7 DNA polymerase, T4 DNA polymerase, T5 DNA polymerase, Klenow class polymerases, Taq polymerase, Pfu DNA polymerase, Vent polymerase, bacteriophage 29, REDTaq™ Genomic DNA polymerase, or sequenase. Exemplary polymerases include, but are not limited to Bacillus stearothermophilus pol I, Thermus aquaticus (Taq) pol I, Pyrccoccus furiosus (Pfu), Pyrococcus woesei (Pwo), Thermus flavus (Tfl), Thermus thermophilus (Tth), Thermus litoris (Tli) and Thermotoga maritime (Tma). These enzymes, modified versions of these enzymes, and combination of enzymes, are commercially available from vendors including Roche, Invitrogen, Qiagen, Stratagene, and Applied Biosystems. Representative enzymes include PHUSION® (New England Biolabs, Ipswich, Mass.), Hot MasterTaq™ (Eppendorf), PHUSION® Mpx (Finnzymes), PyroStart® (Fermentas), KOD (EMD Biosciences), Z-Taq (TAKARA), and CS3AC/LA (KlenTaq, University City, Mo.).

Salts and buffers include those familiar to those skilled in the art, including those comprising MgCl₂, and Tris-HCl and KCl, respectively. Typically, 1.5-2.0 nM of magnesium is optimal for Taq DNA polymerase, however, the optimal magnesium concentration may depend on template, buffer, DNA and dNTPs as each has the potential to chelate magnesium. If the concentration of magnesium [Mg²⁺] is too low, a PCR product may not form. If the concentration of magnesium [Mg²⁺] is too high, undesired PCR products may be seen. In some embodiments the magnesium concentration may be optimized by supplementing magnesium concentration in 0.1 mM or 0.5 mM increments up to about 5 mM.

Buffers used in accordance with the disclosure may contain additives such as surfactants, dimethyl sulfoxide (DMSO), glycerol, bovine serum albumin (BSA) and polyethylene glycol (PEG), as well as others familiar to those skilled in the art. Nucleotides are generally deoxyribonucleoside triphosphates, such as deoxyadenosine triphosphate (dATP), deoxycytidine triphosphate (dCTP), deoxyguanosine triphosphate (dGTP), and deoxythymidine triphosphate (dTTP), which are also added to a reaction adequate amount for amplification of the target nucleic acid. In some embodiments, the concentration of one or more dNTPs (e.g., dATP, dCTP, dGTP, dTTP) is from about 10 μM to about 500 μM which may depend on the length and number of PCR products produced in a PCR reaction.

In some embodiments, the primers used in accordance with the disclosure are modified. The primers may be designed to bind with high specificity to only their intended target (e.g., a particular SNV) and demonstrate high discrimination against further nucleotide sequence differences. The primers may be modified to have a particular calculated melting temperature (Tm), for example a melting temperature ranging from 46° C. to 64° C. To design primers with desired melting temperatures, the length of the primer may be varied and/or the GC content of the primer may be varied. Typically, increasing the GC content and/or the length of the primer will increase the Tm of the primer. Conversely, decreasing the GC content and/or the length of the primer will typically decrease the Tm of the primer. It should be appreciated that the primers may be modified by intentionally incorporating mismatch(es) with respect to the target in order to detect a particular SNV (or other form of sequence non-identity) over another with high sensitivity. Accordingly, the primers may be modified by incorporating one or more mismatches with respect to the specific sequence (e.g., a specific SNV) that they are designed to bind.

In some embodiments, the concentration of primers used in the PCR reaction may be modified or optimized. In some embodiments, the concentration of a primer (e.g., a forward or reverse primer) in a PCR reaction may be, for example, about 0.05 μM to about 1 μM. In particular embodiments, the concentration of each primer is about 1 nM to about 1 μM. It should be appreciated that the primers in accordance with the disclosure may be used at the same or different concentrations in a PCR reaction. For example, the forward primer of a primer pair may be used at a concentration of 0.5 μM and the reverse primer of the primer pair may be used at 0.1 μM. The concentration of the primer may be based on factors including, but not limited to, primer length, GC content, purity, mismatches with the target DNA or likelihood of forming primer dimers.

In some embodiments, the thermal profile of the PCR reaction is modified or optimized. Non-limiting examples of PCR thermal profile modifications include denaturation temperature and duration, annealing temperature and duration and extension time.

The temperature of the PCR reaction solutions may be sequentially cycled between a denaturing state, an annealing state, and an extension state for a predetermined number of cycles. The actual times and temperatures can be enzyme, primer, and target dependent. For any given reaction, denaturing states can range in certain embodiments from about 70° C. to about 100° C. In addition, the annealing temperature and time can influence the specificity and efficiency of primer binding to a particular locus within a target nucleic acid and may be important for particular PCR reactions. For any given reaction, annealing states can range in certain embodiments from about 20° C. to about 75° C. In some embodiments, the annealing state can be from about 46° C. to 64° C. In certain embodiments, the annealing state can be performed at room temperature (e.g., from about 20° C. to about 25° C.).

Extension temperature and time may also impact the allele product yield. For a given enzyme, extension states can range in certain embodiments from about 60° C. to about 75° C.

Quantification of the amounts of the alleles from a quantification assay as provided herein can be performed as provided herein or as otherwise would be apparent to one of ordinary skill in the art. As an example, amplification traces are analyzed for consistency and robust quantification. Internal standards may be used to translate the cycle threshold to amount of input nucleic acids (e.g., DNA). The amounts of alleles can be computed as the mean of performant assays and can be adjusted for genotype.

It has been found that the methods and compositions provided herein can be used to detect low-level nucleic acids, such as non-native nucleic acids, in a sample. Accordingly, the methods provided herein can be used on samples where detection of relatively rare nucleic acids is needed. In some embodiments, any one of the methods provided herein can be used on a sample to detect non-native nucleic acids that are at least about 1% in the sample relative to total nucleic acids, such as total cf-DNA. In some embodiments, any one of the methods provided herein can be used on a sample to detect non-native nucleic acids that are at least about 1.3% in the sample. In some embodiments, any one of the methods provided herein can be used on a sample to detect non-native nucleic acids that are at least about 1.5% in the sample.

Because of the ability to determine amounts of non-native nucleic acids, even at low levels, the methods and compositions provided herein can be used to assess a risk in a subject, such as risk of cancer. A “risk” as provided herein, refers to the presence or absence or progression of any undesirable condition in a subject (such as cancer), or an increased likelihood of the presence or absence or progression of such a condition, e.g., cancer. The cancer can be any one of the cancers provided herein. As provided herein “increased risk” refers to the presence or progression of any undesirable condition in a subject or an increased likelihood of the presence or progression of such a condition. As provided herein, “decreased risk” refers to the absence of any undesirable condition or progression in a subject or a decreased likelihood of the presence or progression (or increased likelihood of the absence or nonprogression) of such a condition.

As provided herein, early detection or monitoring of conditions, such as cancer, can facilitate treatment and improve clinical outcomes. As mentioned above, any one of the methods provided can be performed on a subject at risk of having cancer or a tumor, recurrence of cancer or a tumor or metastasis of a cancer or tumor. Accordingly, in some embodiments, the subject is a subject suspected of having cancer, metastasis, and/or recurrence of cancer or subject having cancer, metastasis and/or recurrence of cancer. In some embodiments, the subject may show no signs or symptoms of having a cancer, metastasis, and/or recurrence. However, in some embodiments, the subject may show symptoms associated with cancer. The type of symptoms will depend upon the type of cancer and are well known in the art.

Cancers include, but are not limited to, hematological cancers, such as leukemias, lymphomas, etc. Cancers include but are not limited to neoplasms, malignant tumors, metastases, or any disease or disorder characterized by uncontrolled cell growth such that it would be considered cancerous. The cancer may be a primary or metastatic cancer.

The risk in a subject, such as in a recipient of a transplant can be determined, for example, by assessing the amount of non-native nucleic acids, such as cf-DNA, and/or the noise, background or discordance QC related to such an assessment. For example, the background, noise or discordance QC can include targets that should not be detected but has a quantity in performing the assay that is greater than zero. In some embodiments, the background, noise or discordance QC can include what would be considered non-informative, erroneous or those that would be expected to be “no calls”. It has been found that subjects with higher non-native fraction of cf-DNA and/or higher background, noise or discordance QC is indicative of cancer risk, such as lymphoma risk.

In some embodiments, any one of the methods provided herein can comprise correlating an increase in the amount of non-native nucleic acids (such as an increase in the ratio, or percentage, of non-native nucleic acids relative to total or native nucleic acids) and/or increased background, noise or discordance QC, with an increased risk of a condition, such as cancer. In some embodiments of any one of the methods provided herein, correlating comprises comparing an amount or level (e.g., concentration, ratio or percentage) of one or more of the indicators as provided herein to a threshold value to identify a subject at increased or decreased risk of a condition. In some embodiments of any one of the methods provided herein, a subject having an increased amount of non-native nucleic acids and/or background, noise or discordance QC compared to a threshold value is identified as being at increased risk of one or more cancers. In some embodiments of any one of the methods provided herein, a subject having a decreased or similar amount of non-native nucleic acids and/or background, noise or discordance QC compared to a threshold value is identified as being at decreased risk of one or more cancers.

As used herein, “amount” refers to any quantitative value for the measurement of nucleic acids and can be given in an absolute or relative amount. Further, the amount can be a total amount, frequency, ratio, percentage, etc. As used herein, the term “level” can be used instead of “amount” but is intended to refer to the same types of values.

“Threshold” or “threshold value”, as used herein, refers to any predetermined level or range of levels that is indicative of the presence or absence of a condition or the presence or absence of a risk. The threshold value can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quadrants, the lowest quadrant being subjects with the lowest risk and the highest quadrant being subjects with the highest risk. The threshold value can depend upon the particular population selected. For example, an apparently healthy population will have a different ‘normal’ range. As another example, a threshold value can be determined from baseline values before the presence of a condition or risk or after a course of treatment. Such a baseline can be indicative of a normal or other state in the subject not correlated with the risk or condition that is being tested for. In some embodiments, the threshold value can be a baseline value of the subject being tested. Accordingly, the predetermined values selected may take into account the category in which the subject falls. Appropriate ranges and categories can be selected with no more than routine experimentation by those of ordinary skill in the art. In one embodiment of any one of the methods provided herein, the threshold is any of the thresholds provided herein.

Changes in the levels of non-native nucleic acids and/or background, noise or discordance QC can also be monitored over time. For example, a change from a threshold value (such as a baseline) in the amount, such as ratio or percentage, of non-native nucleic acids and/or background, noise or discordance QC can be used as a non-invasive clinical indicator of risk. e.g., risk associated with cancer. This can allow for the measurement of variations in a clinical state and/or permit calculation of normal values or baseline levels. Generally, as provided herein, the amount, such as the ratio or percent, of non-native nucleic acids and/or background, noise or discordance QC can be indicative of the presence or absence of a risk associated with a condition, such as risk associated with cancer, or can be indicative of the need for further testing or surveillance. In one embodiment of any one of the methods provided herein, the method may further include an additional test(s) for assessing a condition, such as cancer, etc. The additional test(s) may be any one of the methods provided herein.

In some embodiments of any one of the methods provided herein, where a non-native nucleic acid amount, such as ratio or percentage, and/or background, noise or discordance QC is determined to be above a threshold value, any one of the methods provided herein can further comprise performing another test on the subject or sample therefrom. Such other tests can be any other test known by one of ordinary skill in the art to be useful in determining the presence or absence of a risk, e.g., in a subject having or suspected of having cancer, progressing cancer, a metastasis or recurrence of cancer, etc. In some embodiments, the other test is any one of the methods provided herein.

Exemplary additional tests for subjects suspected of having cancer, metastasis, and/or recurrence, include, but are not limited to, biopsy (e.g., fine-needle aspiration, core biopsy, or lymph node removal), X-ray, CT scan, ultrasound, MRI, endoscopy, circulating tumor cell levels, complete blood count, detection of specific tumor biomarkers (e.g., EGFR,ER, HER2, KRAS, c-KIT, CD20, CD30, PDGFR, BRAF, or PSMA), and/or genotyping (e.g., BRCA1, BRCA2, HNPCC, MLH1, MSH2, MSH6, PMS1, or PMS2). The type of additional test(s) will depend upon the type of suspected cancer/metastasis/recurrence and is well within the determination of the skilled artisan.

As provided herein, any one of the methods provided can include a step of providing a therapy or information regarding a therapy to a subject. In some of these embodiments, the therapy is a cancer treatment. In some embodiments, the information is provided in written form or electronic form. In some embodiments, the information may be provided as computer-readable instructions. In some embodiments, the information may be provided orally.

The therapies can be for treating cancer, a tumor or metastasis, such as an anti-cancer therapy. Such therapies include, but are not limited to, antitumor agents, such as docetaxel; corticosteroids, such as prednisone or hydrocortisone; immunostimulatory agents; immunomodulators; or some combination thereof. Antitumor agents include cytotoxic agents, chemotherapeutic agents and agents that act on tumor neovasculature. Cytotoxic agents include cytotoxic radionuclides, chemical toxins and protein toxins. The cytotoxic radionuclide or radiotherapeutic isotope can be an alpha-emitting or beta-emitting. Cytotoxic radionuclides can also emit Auger and low energy electrons. Suitable chemical toxins or chemotherapeutic agents include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Other antineoplastic agents include dolastatins (U.S. Pat. Nos. 6,034,065 and 6,239,104) and derivatives thereof. Toxins also include poisonous lectins, plant toxins such as ricin, abrin, modeccin, botulina and diphtheria toxins. Other chemotherapeutic agents are known to those skilled in the art. Examples of cancer chemotherapeutic agents include, but are not limited to, irinotecan (CPT-11); erlotinib; gefitinib (Iressa™); imatinib mesylate (Gleevec); oxalipatin; anthracyclins-idarubicin and daunorubicin; doxorubicin; alkylating agents such as melphalan and chlorambucil; cis-platinum, methotrexate, and alkaloids such as vindesine and vinblastine. In some embodiments, further or alternative cancer treatments are contemplated herein, such as radiation and/or surgery.

Administration of a treatment or therapy may be accomplished by any method known in the art (see, e.g., Harrison's Principle of Internal Medicine, McGraw Hill Inc.). Preferably, administration of a treatment or therapy occurs in a therapeutically effective amount. Administration may be local or systemic. Administration may be parenteral (e.g., intravenous, subcutaneous, or intradermal) or oral. Compositions for different routes of administration are well known in the art (see, e.g., Remington's Pharmaceutical Sciences by E. W. Martin).

In still other embodiments, any one of the methods can be used to assess the efficacy of a therapy (or treatment) where improved values can indicate less of a need for the therapy, while worsening values can indicate the need for a therapy, a different therapy, or an increased amount of a therapy. Any one of the methods provided herein can include the step of evaluating the need or dose of a therapy based on the result of one or more comparisons at one or more time points.

In some embodiments, the method may further comprise further testing or recommending further testing to the subject and/or treating or suggesting treatment to the subject. In some of these embodiments, the further testing is any one of the methods provided herein.

It may be particularly useful to a clinician to have a report that contains the value(s) provided herein. In one aspect, therefore such reports are provided. Reports may be in oral, written (or hard copy) or electronic form, such as in a form that can be visualized or displayed. In some embodiments, the “raw” results for each assay as provided herein are provided in a report, and from this report, further steps can be taken to analyze the amount(s) of non-native nucleic acids and/or background, noise or discordance QC. In other embodiments, the report provides multiple values for the amounts and/or background, noise or discordance QC, such as over time. From the amounts and/or background, noise or discordance QC, in some embodiments, a clinician may assess the need for a treatment for the subject or the need to monitor the subject.

Accordingly, in any one of the methods provided herein, the method can include assessing the amount of non-native nucleic acids in the subject and/or background, noise or discordance QC at another point in time or times. Such assessing can be performed with any one of the methods provided herein.

Any one of the methods provided herein can comprise extracting nucleic acids, such as cell-free DNA, from a sample obtained from a subject, such as a subject at risk of, or having, cancer. Such extraction can be done using any method known in the art or as otherwise provided herein (see, e.g., Current Protocols in Molecular Biology, latest edition, or the QIAamp circulating nucleic acid kit or other appropriate commercially available kits). An exemplary method for isolating cell-free DNA from blood is described. Blood containing an anti-coagulant such as EDTA or DTA is collected from a subject. The plasma, which contains cf-DNA, is separated from cells present in the blood (e.g., by centrifugation or filtering). An optional secondary separation may be performed to remove any remaining cells from the plasma (e.g., a second centrifugation or filtering step). The cf-DNA can then be extracted using any method known in the art, e.g., using a commercial kit such as those produced by Qiagen. Other exemplary methods for extracting cf-DNA are also known in the art (see, e.g., Cell-Free Plasma DNA as a Predictor of Outcome in Severe Sepsis and Septic Shock. Clin. Chem. 2008, v. 54, p. 1000-1007; Prediction of MYCN Amplification in Neuroblastoma Using Serum DNA and Real-Time Quantitative Polymerase Chain Reaction. JCO 2005, v. 23, p. 5205-5210; Circulating Nucleic Acids in Blood of Healthy Male and Female Donors. Clin. Chem. 2005, v. 51, p. 1317-1319; Use of Magnetic Beads for Plasma Cell-free DNA Extraction: Toward Automation of Plasma DNA Analysis for Molecular Diagnostics. Clin. Chem. 2003, v. 49, p. 1953-1955; Chiu R W K, Poon L L M, Lau T K, Leung T N, Wong E M C, Lo Y M D. Effects of blood-processing protocols on fetal and total DNA quantification in maternal plasma. Clin Chem 2001; 47:1607-1613; and Swinkels et al. Effects of Blood-Processing Protocols on Cell-free DNA Quantification in Plasma. Clinical Chemistry, 2003, vol. 49, no. 3, 525-526).

In some embodiments of any one of the methods provided herein, a pre-amplification step is performed. An exemplary method of such an amplification is as follows, and such a method can be included in any one of the methods provided herein. Approximately 15 ng of cell free plasma DNA is amplified in a PCR using Q5 DNA polymerase with approximately 100 targets where pooled primers were at 6 uM total. Samples undergo approximately 35 cycles. Reactions are in 25 ul total. After amplification, samples can be cleaned up using several approaches including AMPURE bead cleanup, bead purification, or simply ExoSAP-IT™ it, or Zymo.

As used herein, the sample from a subject can be a biological sample. Examples of such biological samples include whole blood, plasma, serum, urine, etc. In some embodiments of any one of the methods provided herein, addition of further nucleic acids, e.g., a standard, to the sample can be performed.

The present disclosure also provides methods for determining a plurality of SNV targets for use in any one of the methods provided herein or from which any one of the compositions of primers can be derived. A method of determining a plurality of SNV targets, in some embodiments comprises a) identifying a plurality of highly heterozygous SNVs in a population of individuals, b) designing one or more primers spanning each SNV, c) selecting sufficiently specific primers, d) evaluating multiplexing capabilities of primers, such as at a common melting temperature and/or in a common solution, and e) identifying sequences that are evenly amplified with the primers or a subset thereof.

As used herein, “highly heterozygous SNVs” are those with a minor allele at a sufficiently high percentage in a population. In some embodiments, the minor allele is at least 25%, 26%, 27%, 28%, 29%, 30%, 31%, 32%, 33%, 34% or 35% or more in the population. In any one of these embodiments, the minor allele is less than 50%, 49%, 45%, 44%, 43%, 42%, 41%, or 40% in the population. Such SNVs increase the likelihood of providing a target that is different between the native and non-native nucleic acids.

Primers can be designed to generally span a 70 bp window but some other window may also be selected, such as one between 65 bps and 75 bps, or between 60 bps and 80 bps. Also, generally, it can be desired for the SNV to fall about in the middle of this window. For example, for a 70 bp window, the SNV can be between bases 10-60 or 20-50, such as between bases 30-40. The primers as provided herein can be designed to be adjacent to the SNV.

As used herein, “sufficiently specific primers”, were those that demonstrated discrimination between amplification of the intended allele versus amplification of the unintended allele. Thus, with PCR a cycle gap can be desired between amplification of the two. In one embodiment, the cycle gap can be at least a 5, 6, 7 or 8 cycle gap.

Further, sequences can be selected based on melting temperatures, generally those with a melting temperature of between 45-55 degrees C. were selected as “moderate range sequences”. Other temperature ranges may be desired and can be determined by one of ordinary skill in the art. A “moderate range sequence” generally is one that can be amplified in a multiplex amplification format within the temperature. In some embodiments, the GC % content can be between 30-70% or between 35%-65%, such as between 33-66%.

In one embodiment of any one of the methods provided herein, the method can further comprise excluding sequences associated with difficult regions. “Difficult regions” are any regions with content or features that make it difficult to reliably make predictions about a target sequence or are thought to not be suitable for multiplex amplification. Such regions include syndromic regions, low complexity regions, regions with high GC content or that have sequential tandem repeats. Other such features can be determined or are otherwise known to those of ordinary skill in the art.

The present disclosure also provides compositions or kits that can be useful for assessing an amount of non-native nucleic acids in a sample and/or noise, background or discordance QC. In some embodiments, the composition or kit comprises a plurality of primer pairs. Each of the primer pairs of the composition or kit can comprise a forward and a reverse primer, wherein there is a 3′ mismatch in one of the primers (e.g., at the penultimate 3′ nucleotide) in some embodiments of any one of the methods, compositions or kits provided herein. In some embodiments of any one of the methods, compositions or kits provided herein, this mismatch is at a 3′ nucleotide and adjacent to the SNV position and when the particular SNV is not present there is a double mismatch with respect to the other allele of the SNV target. In some embodiments of any one of the methods, compositions or kits provided herein, the mismatch primer of a primer pair is the forward primer. In some embodiments of any one of the methods, compositions or kits provided herein, the reverse primer for each allele of a SNV target is the same.

In some embodiments of any one of the compositions or kits provided herein, the composition or kit comprises primer pairs for at least 18, 21, 24, 27, 30, 33, 36, 39, 42, 45, 48, 51, 54, 57, 60, 63, 66, 69, 72, 75, 78, 81, 84 or 87 targets. In some embodiments of any one of the compositions or kits, the compositions or kits comprise primer pairs for fewer than 96, 93, 90, 87, 84, 81, 78, 75, 72 or 69 targets. In some embodiments, primer pairs for 18-30, 18-45, 18-60, 18-75, 18-80, 18-85, 18-90 or 18-95 targets are included in any one of the compositions or kits. In some embodiments primer pairs for 21-30, 21-45, 21-60, 21-75, 21-80, 21-85, 21-90 or 21-95 targets are included. In some embodiments, primer pairs for 24-30, 24-45, 24-60, 24-75, 24-80, 24-85, 24-90 or 24-95 targets are included. In some embodiments primer pairs for 30-45, 30-60, 30-75, 30-80, 30-85, 30-90 or 30-95 targets are included. In some embodiments of any one of the methods or compositions provided herein, primer pairs for 40-45, 40-60, 40-75, 40-80, 40-85, 40-90 or 40-95 targets are included. In some embodiments primer pairs for 45-60, 45-75, 45-80, 45-85, 45-90 or 45-95 targets are included. In some embodiments primer pairs for 50-60, 50-75, 50-80, 50-85, 50-90 or 50-95 targets are included.

In some embodiments of any one of the compositions or kits, the compositions or kits comprise primer pairs for at least 6 informative targets. In some embodiments the composition or kit comprises primer pairs for at least 6 but less than 35 informative targets, less than 30 informative targets, less than 25 informative targets, less than 20 informative targets, less than 15 informative targets, or less than 10 informative targets.

In other embodiments of the compositions or kits provided herein, one or more, including all, of the primer pairs are not for cancer-specific SNV targets. In some embodiments of any one of the compositions or kits provided herein, the primer pairs of the composition or kit are designed to be compatible for use in amplification-based quantification assay, such as a quantitative PCR assay. For example, the primer pairs are designed to prevent primer dimers and/or limit the number of off-target binding sites. It should be appreciated that the primer pairs of the composition or kit may be optimized or designed in accordance with any one of the methods described herein.

In some embodiments, any one of the compositions or kits provided further comprises a buffer. In some embodiments, the buffers contain additives such as surfactants, dimethyl sulfoxide (DMSO), glycerol, bovine serum albumin (BSA) and polyethylene glycol (PEG) or other PCR reaction additive. In some embodiments, any one of the compositions or kits provided further comprises a polymerase for example, the composition or kit may comprise E. coli DNA polymerase, Klenow fragment of E. coli DNA polymerase I, T7 DNA polymerase, T4 DNA polymerase, T5 DNA polymerase, Klenow class polymerases, Taq polymerase, Pfu DNA polymerase. Vent polymerase, bacteriophage 29, REDTaq™ Genomic DNA polymerase, or sequenase. In some embodiments, any one of the compositions or kits provided further comprises one or more dNTPs (e.g., dATP, dCTP, dGTP, dTTP). In some embodiments, any one of the compositions or kits provided further comprises a probe (e.g., a TAQMAN™ probe).

A “kit,” as used herein, typically defines a package or an assembly including one or more of the compositions of the invention, and/or other compositions associated with the invention, for example, as previously described. Any one of the kits provided herein may further comprise at least one reaction tube, well, chamber, or the like. Any one of the primers, primer systems (such as a set of primers for a plurality of targets) or primer compositions described herein may be provided in the form of a kit or comprised within a kit.

Each of the compositions of the kit may be provided in liquid form (e.g., in solution), in solid form (e.g., a dried powder), etc. A kit may, in some cases, include instructions in any form that are provided in connection with the compositions of the invention in such a manner that one of ordinary skill in the art would recognize that the instructions are to be associated with the compositions of the invention. The instructions may include instructions for performing any one of the methods provided herein. The instructions may include instructions for the use, modification, mixing, diluting, preserving, administering, assembly, storage, packaging, and/or preparation of the compositions and/or other compositions associated with the kit. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different from illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The following description provides examples of the methods provided herein.

### EXAMPLES

### Example 1—MOMA Assay with Genotype Information

### SNV Target Selection

Identification of targets for multiplexing in accordance with the disclosure may include one or more of the following steps, as presently described. First, highly heterozygous SNPs can be screened on several ethnic control populations (Hardy-Weinberg p>0.25), excluding known difficult regions. Difficult regions include syndromic regions likely to be abnormal in patients and regions of low complexity, including centromeres and telomeres of chromosomes. Target fragments of desired lengths can then be designed in silico. Specifically, two 20-26 bp primers spanning each SNP's 70 bp window can be designed. All candidate primers can then be queried to GCRh37 using BLAST. Those primers that were found to be sufficiently specific can be retained, and monitored for off-target hits, particularly at the 3′ end of the fragment. The off-target candidate hits can be analyzed for pairwise fragment generation that would survive size selection. Selected primers can then be subjected to an in silico multiplexing evaluation. The primers' computed melting temperatures and guanine-cytosine percentages (GC %) can be used to filter for moderate range sequences. An iterated genetic algorithm and simulated annealing can be used to select candidate primers compatible for 400 targets, ultimately resulting in the selection of 800 primers. The 800 primers can be generated and physically tested for multiplex capabilities at a common melting temperature in a common solution. Specifically, primers can be filtered based on even amplification in the multiplex screen and moderate read depth window. Forty-eight assays can be designed for MOMA using the top performing multiplexed SNPs. Each SNP can have a probe designed in WT/MUT at four mismatch choices; eight probes per assay. The new nested primers can be designed within the 70 bp enriched fragments. Finally, the primers can be experimentally amplified to evaluate amplification efficiency (8 probes x 48 assays in triplicate, using TAQMAN™)

### A Priori Genotyping Informativeness of Each Assay

Using, for example, known or possible native and non-native genotypes at each assayed SNP, a subset of informative assays was selected. Note that subject homozygous sites can be used where the non-native is any other genotype. Additionally, if the non-native genotype is not known, it can be inferred. Genotypes may also be learned through sequencing, SNP microarray, or application of a MOMA assay on known 0% (clean recipient) samples.

### Post Processing Analysis of Multiplex Assay Performance

Patient-specific MOMA probe biases can be estimated across an experimental cohort. Selection iteratively can be refined to make the final non-native percent call.

### Reconstruction Experiment

The sensitivity and precision of the assay can be evaluated using reconstructed plasma samples with known mixing ratios. Specifically, the ratios of 1:10, 1:20, 1:100, 1:200, and 1:1000 can be evaluated. Generally, primers for 95 SNV targets can be used as described herein in some embodiments.

### Example 2—MOMA Assay with Native (Subject) but not Non-Native Genotype Information

### Expectation Maximization Method

To work without non-native genotype information, the following procedure may be performed to infer informative assays and allow for quantification of non-native-specific cell-free DNA in plasma samples. All assays can be evaluated for performance in the full information scenario. This procedure thus assumed clean AA/AB/BB genotypes at each assay and unbiased behavior of each quantification. With native genotype, assays known to be homozygous in the subject can be selected. Contamination can be attributed to the non-native nucleic acids, and the assay collection created a tri-modal distribution with three clusters of assays corresponding to the non-, half, and fully-informative assays. With sufficient numbers of recipient homozygous assays, the presence of non-native fully informative assays can be assumed.

If the native genotype is homozygous and known, then if a measurement that is not the non-native genotype is observed, the probes which are truly non-native-homozygous will have the highest cluster and equal the guess whereas those that are non-native heterozygous will be at half the guess. A probability distribution can be plotted and an expectation maximization algorithm (EM) can be employed to infer non-native genotype. Such can be used to infer the non-native genotype frequency in any one of the methods provided herein.

Accordingly, an EM algorithm was used to infer the most likely non-native genotypes at all assayed SNV targets. With inferred non-native genotypes, quantification may proceed as in the full-information scenario. EM can begin with the assumption that the minor allele ratio found at an assay follows a tri-modal distribution, one for each combination of subject and non-native, given all assays are “AA” in the subject (or flipped from “BB” without loss of generality). With all non-native genotypes unknown, it is possible to bootstrap from the knowledge that any assays exhibiting nearly zero minor allele are non-native AA, and the highest is non-native BB. Initial guesses for all non-native genotypes were recorded, and the mean of each cluster calculated. Enforcing that the non-native BB assays' mean is twice that of the non-native AB restricts the search. The algorithm then reassigns guessed non-native genotypes based on the clusters and built-in assumptions. The process was iterative until no more changes were made (e.g., the dd-ctDNA % is recalculated until convergence). The final result is a set of the most likely non-native genotypes given their measured divergence from the background. Generally, every target falls into the model; a result may be tossed if between groups after maximization.

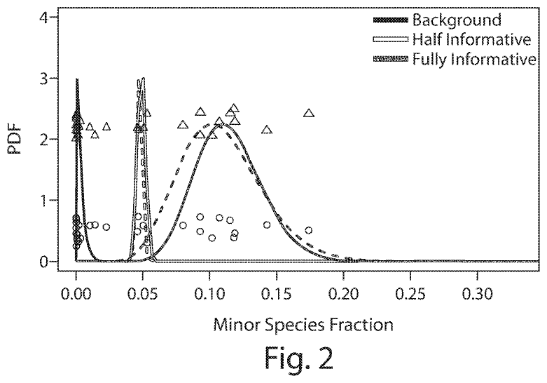

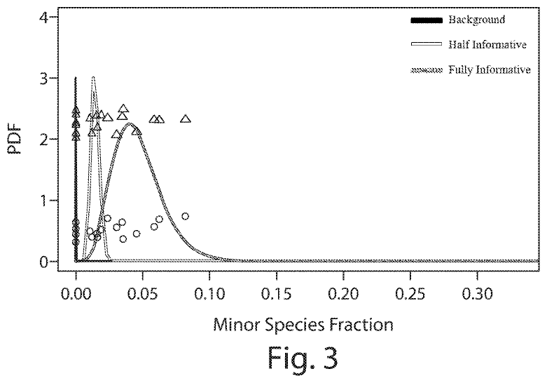

FIGS. 2 and 3 show exemplary results from plasma samples handled in this manner. The x-axis is the donor % (non-native %) for any assay found native (subject) homozygous. The rows of points represent individual PCR assay results. The bottom-most row of circles represents the initial guess of non-native genotypes, some AA, some A/B and some BB. Then the solid curves were drawn representing beta distributions centered on the initial assays, dotted for homozygous (fully informative) and white for heterozygous (half informative) with black curves representing the distribution of non-informative assays or background noise. The assays were re-assigned updated guesses in the second row. The second row's curves use dashed lines. The top row is the final estimate because no change occurred. Double the peak of the white dashed curve corresponds to the maximum likelihood non-native % call, at around 10%, or equal to the mean of the dotted curve.

A reconstruction experiment (Recon1) using DNA from two individuals was created at 10%, 5%, 1%, 0.5%, and 0.1%. All mixes were amplified with a multiplex library of targets, cleaned, then quantitatively genotyped using a MOMA method. The analysis was performed with genotyping each individual in order to know their true genotypes. Informative targets were determined using prior knowledge of the genotype of the major individual (looking for homozygous sites), and where the second individual was different, and used to calculate fractions (percentage) using informative targets. The fractions were then calculated (depicted in black to denote “With Genotype” information).

A second reconstruction experiment (Recon2), beginning with two individuals, major and minor, was also created at 10%, 5%, 1%, 0.5%, and 0.1%. All mixes were amplified with the multiplex library of targets, cleaned, and then quantitatively genotyped using a MOMA method. The analysis was performed by genotyping each individual in order to know their true genotypes. Informative targets were determined using prior knowledge of the genotype of the second individual as described above. The fractions were then calculated (depicted in black to denote “With Genotype” information).

These reconstructions were run again the next day (Recon3).

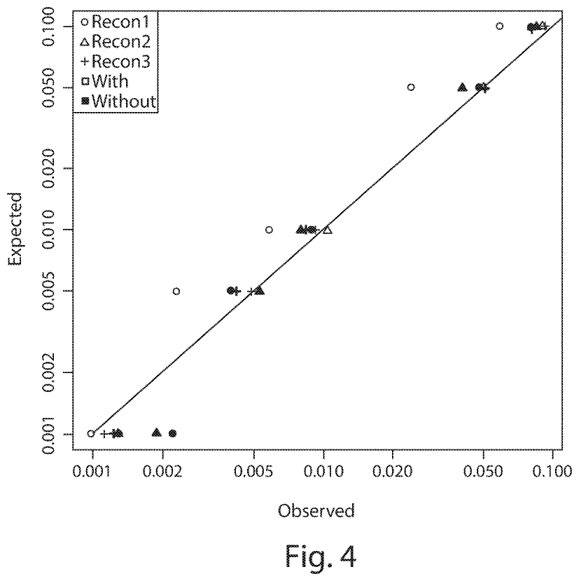

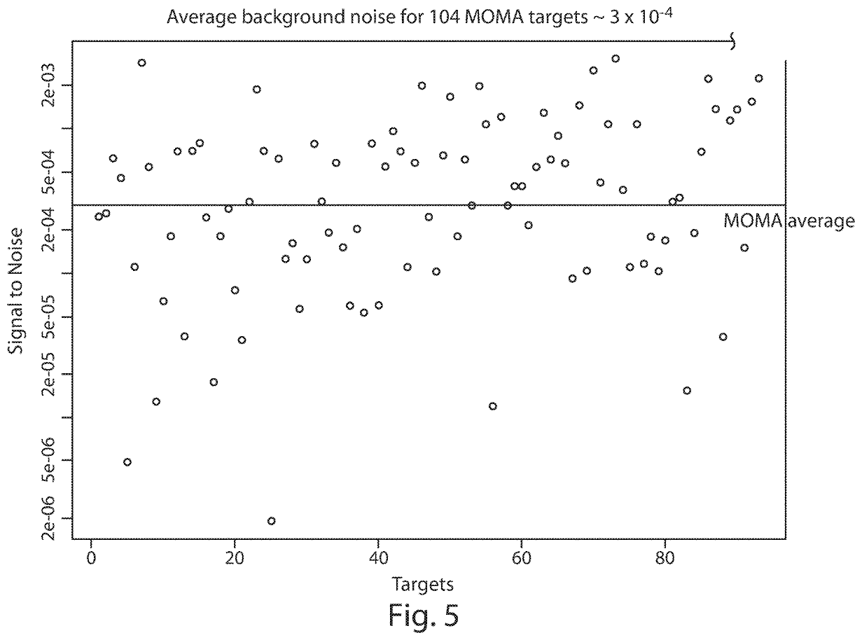

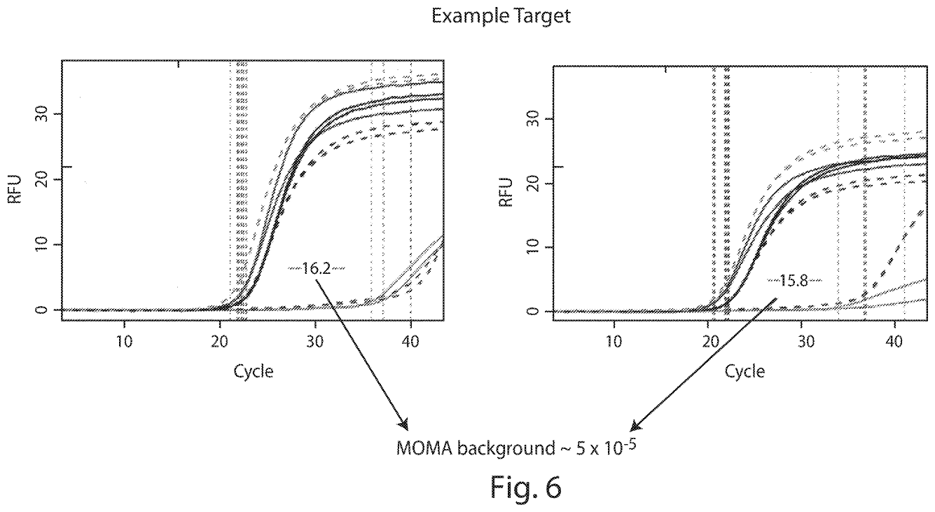

The same reconstruction samples (Recon 1,2,3) were then analyzed again only using the genotyping information available for the first individual (major DNA contributor). Genotyping information from the second individual (minor DNA contributor) was not used. Approximately 38-40 targets were used to calculate fractions without genotyping (simulating without donor); they are presented as shaded points (FIG. 4). It was found that each target that was native (subject) homozygous was generally useful. The circles show a first estimate, a thresholding; those on the right were thought to be fully informative and those on the left, not. The triangles along the top were the same targets, but for the final informativity decisions they were recolored.

Alternative MOMA Assay with Native (Subject) but not Non-Native Genotype Information

In contrast to expectation-maximization, MOMA assays may be performed knowing only the native (subject) genotype and using discordance quality check (“dQC”), an additional metric. The method evaluates all possible non-native (donor) genotypes, reports what the input non-native cfDNA % could have been, and then is refined by statistical analysis of model performance to select the more-likely genotypes when they become apparent.

Using the native genotype, targets are selected as candidates for informativity. The subset of those deemed to be robustly quantifiable using normal analytic quality checks are collected. Without loss of generality, any sites know to be recipient homozygous for VAR ‘down’, are excluded and all recipient homozygous for REF targets are used. The result is between five and 30 targets cleanly reporting a non-native cfDNA % in one of three possible categories: non informative, fully informative, and half informative (corresponding to the unknown non-native genotypes RR, VV, and RV). All possible outcomes of the sample are analyzed by randomizing the non-native genotype candidates in parallel, and evaluating thousands of potential outcomes. The aggregate and 95% confidence interval of the ‘believable’ resimulations are reported as a non-native cfDNA upper and lower bound.

The result of each resimulation is a three-dimensional point. Each possible non-native genotype set results in three numeric outcomes: 1) the primary non-native cfDNA call, the median of targets, 2) the robust coefficient of variation (rCV), a measure of target-model consistency, and 3) the discordance quality check (dQC). If the sample and chemistry were perfectly sensitive and specific, the unique genotype combination corresponding to minimal dQC and minimal rCV would be the correct selection.

The “rCV” metric is similar to a simple coefficient of variation (CoV), in that it serves to indicate how tightly the separate target measures cluster around their center. Where CoV uses standard deviation divided by mean, rCV uses a ‘robust standard deviation’ divided by an offset median. The robust standard deviation is a median of absolute differences from the median of a sample, with an augment scalar to approximate the Gaussian distribution's standard deviation. The denominator for the rCV is offset by a quarter of one percent to stabilize values near zero. High values of rCV can indicate incorrectly assigned genotypes.

A complete enumeration of non-native genotypes is not feasible due to the approximately 3\hat{ }30 combinations. Also, the true genotype selection may not correspond to the extreme-lowest rCV/dQC simulation. Instead, a Monte-Carlo simulation can be used to approximate the distribution. For the average sample, 10,000 resimulations will yield a good spread of potential outcomes. Selecting those with below-median dQC and rCV corresponds to the more ‘well behaved’ simulations and their 95% confidence interval will capture the true non-native cfDNA %.

The first step of the method is a short simulation of 3,000 genotype combinations with naïve selections. Each target is assigned one of four genotypes (RR, RV, VV, N/A) with probability (25/110, 50/110, 25/110, 10/110), corresponding to a Hardy-Weinberg style ¼-½-¼ plus a tenth chance of no-call at each target. The samples are calculated normally, yielding 3,000 triplet values and a large genotypes selection matrix.

The second phase looks for obvious correlations by building a linear model between the rCV and the selected genotypes, and a second linear model with dQC as outcome. In each linear modeling, the target sequences (genomic loci) with strong coefficients (greater than their standard deviation and a floor) are identified. Targets which, when selected as homozygous REF significantly reduce the rCV and dQC are be assumed to be truly non-native:RR. The same is true for VV (the targets are assumed to be non-native: VV). Using linear modeling allows the heterozygous selections to contribute half information. A beliefs vector (length=n_targets) is built up with zeroes for uncertain loci and plus or minus ten at now-known loci.

The third phase collects the portion of the 3,000 initial simulations with very high dQC. “Very high” in this context is half the maximum, as it is a skewed distribution with most simulations near their minimum. The dQC is high when the non-informative targets are predominantly calling values higher than zero/background, so these selections can correspond to incorrect assignments. A weights matrix tallies the genotype selections of this subset. Using Shannon Entropy, the targets that are almost always showing one genotype in these simulations are detected. These have a weight penalty applied to the beliefs vector. Similarly, the genotypes that are rarely chosen in this subset are noted and given a weight-bonus on the beliefs vector. Specifically, when entropy is below 1, the chosen genotype is selected by the high-background subset, and when the selection is less than five percent, it is considered rarely chosen.

The fourth phase is a larger resimulation at 30,000 trials incorporating the new beliefs vector. In each trial, two elements of the beliefs vector are ignored to ensure diversity of outcomes. The 30,000 outcomes are analyzed for the more “well-behaved” quadrant, and become the outcome.

The fifth phase is the outcome data selection. Simulations corresponding to the fishtail phenomenon are identified. Considering that the true (unknown) non-native genotypes could be mostly informative, or mostly not-informative, the true distribution can vary significantly. Depending on how many actually-zero targets are involved, the outcome distribution will become skewed towards reporting zero percent when these targets are taken as possibly informative. On a patient-to-patient basis, some samples have a long tail of resimulation outcomes and a wide non-native cfDNA 95% CI, reaching toward zero. These resimulations are recognized as a property of the rCV and median metrics involved, and a hard back-wall function is prepared to exclude recognizably incorrect combinations. Nominally backwall-identified resimulations are ignored for further analysis, unless they constitute >95% of the sample, in which case the true non-native cfDNA value may be in the background noise and they are included.

Resimulations in the lower quartile of dQC after excluding backwall are considered the lower-background selections. Resimulations among the lower-background in the lower third of rCV are considered acceptable CV. The acceptable CV resimulations with lower background and not backwall are considered good choices among which the final outputs are computed. The 2.5th and 97.5th percentiles of these ‘good choices’ are the final outcome of the strategy, and the higher value or an average may be used.

The method skips all the possible error modes in collecting donor genotype data. For example, among 570 research samples, the correlation is 98% between this method and that described in Example 1.

### Example 3—MOMA Cf-DNA Assay

### Principles and Procedures of a MOMA Cf-DNA Assay

This exemplary assay is designed to determine the percentage of non-native cf-DNA present in a subject's blood sample. In this embodiment, the subject's blood sample is collected in an EDTA tube and centrifuged to separate the plasma and buffy coat. The plasma and buffy coat can be aliquoted into two separate 15 mL conical tubes and frozen. The plasma sample can be used for quantitative genotyping (qGT), while the buffy coat can be used for basic genotyping (bGT) of the subject.

The first step in the process can be to extract cell free DNA from the plasma sample (used for qGT) and genomic DNA (gDNA) from the buffy coat, whole blood, or tissue sample (used for bGT). The total amount of cfDNA can be determined by qPCR and normalized to a target concentration. This process is known as a cfDNA Quantification. gDNA can be quantified using UV-spectrophotometry and normalized.

The normalized patient DNA can be used as an input into a highly-multiplexed library PCR pre-amplification reaction containing, for example, 96 primer pairs, each of which amplify a region including one of the MOMA target sites. The resulting library can be used as the input for either the bGT or qGT assay as it consists of PCR amplicons having the MOMA target primer and probe sites. This step can improve the sensitivity of the overall assay by increasing the copy number of each target prior to the highly-specific qPCR amplification. Controls and calibrators/standards can be amplified with the multiplex library alongside patient samples. Following the library amplification, an enzymatic cleanup can be performed to remove excess primers and unincorporated deoxynucleotide triphosphates (dNTPs) to prevent interference with the downstream amplification.

In a parallel workflow, the master mixes can be prepared and transferred to a 384-well PCR plate. The amplified samples, controls, and calibrators/standards can then be diluted with the library dilution buffer to a predetermined volume and concentration. The diluted samples and controls can be aliquoted to a 6-well reservoir plate and transferred to the 384-well PCR plate using an acoustic liquid handler. The plate can then be sealed and moved to a real-time PCR amplification and detection system.

MOMA can perform both the basic and quantitative genotyping analyses by targeting biallelic SNPs that are likely to be distinct between non-native and subject genomes, making them highly informative. The basic genotyping analysis can label the non-native and native (subject) SNVs with three possible genotypes at each target (e.g., homozygous REF, heterozygous REF and VAR, and homozygous VAR). This information can be used for the quantitative genotyping analysis, along with standard curves, to quantitate to the allele ratio for each target, known as a minor-species proportion. The median of all informative and quality-control passed allele ratios can be used to determine the % of non-native cf-DNA.

### Example 4—Use of Discordant Quality Control (dQC) for Sample Analysis

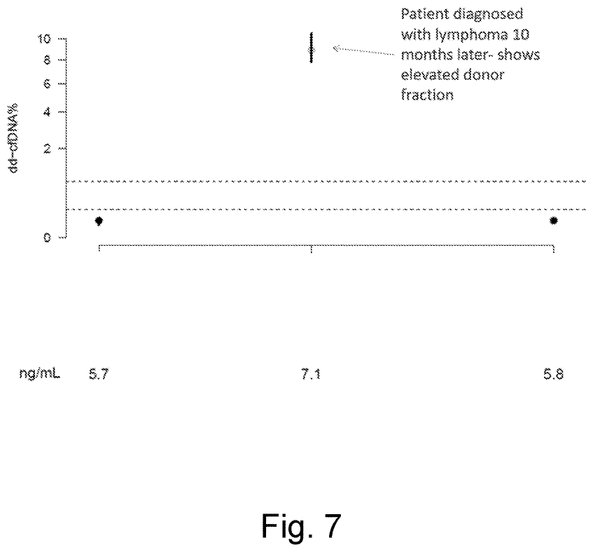

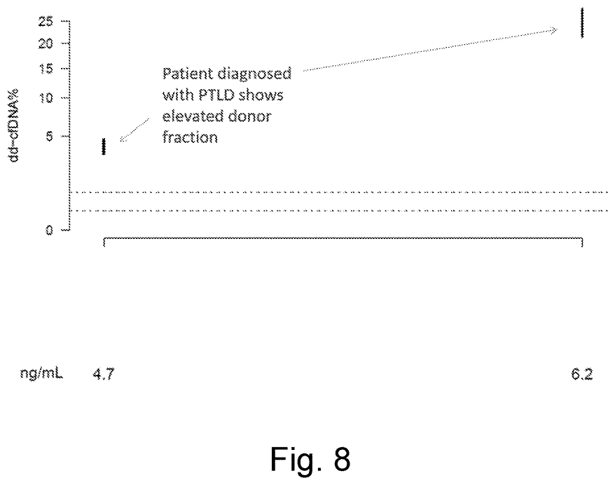

Samples from patients who had undergone transplants were analyzed after their respective transplants. In samples with noise, background or discordance QC levels greater than 5%, an instance of lymphoma (FIG. 7) and a case of post-transplant lymphoproliferative disorder (PTLD) (FIG. 8) were found. In both cases, the patients showed elevated non-native cfDNA percentages as well.

In 292 samples from 86 subjects, the average dQC was 1.62% in samples with lymphoma or post-transplant lymphoproliferative disorders (PTLD) (n=8), whereas the average dQC was 0.32% in samples without cancer (n=284) (p<0.01). The results of the samples with lymphoma or PTLD are shown in Table 1 below.

TABLE 1
Comparison of Method 2 (non-native genotype unknown) and Method
1 (non-native genotype known) Discordance QC

| | Method | Method | | Method 1 | Method2. | Cancer |
|---|---|---|---|---|---|---|
| Subject | 2 Avg | 2 rCV | Method1 | Discordance.QC | Discordance.QC | Type |
| Subject 1 | 0.12% | 0.2416 | 0.070% | 0.02% | 0.03% | Lymphoma |
| Subject 1* | 9.26% | 0.4615 | 8.930% | 6.72% | 4.91% | Lymphoma |
| Subject 1# | 0.10% | 0.2015 | 0.070% | 0.03% | 0.04% | Lymphoma |
| Subject 2 | 0.28% | 0.2644 | 0.200% | 0.02% | 0.07% | PTLD |
| Subject 2 | 0.14% | 0.1973 | 0.110% | 0.02% | 0.05% | PTLD |
| Subject 2 | 0.13% | 0.2857 | 0.120% | 0.03% | 0.05% | PTLD |
| Subject 3 | 24.04% | 0.4243 | NA | NA | 6.24% | PTLD |
| Subject 3 | 3.94% | 0.606 | NA | NA | 1.60% | PTLD |

*Subject was diagnosed with B cell lymphoma 10 months after sample draw. High dQC by both Method 1 and Method 2.
#patient completed therapy in 4 months before this sample draw. dQC low by both Method 1 and Method 2.

### Example 5—Examples of Computer-Implemented Embodiments

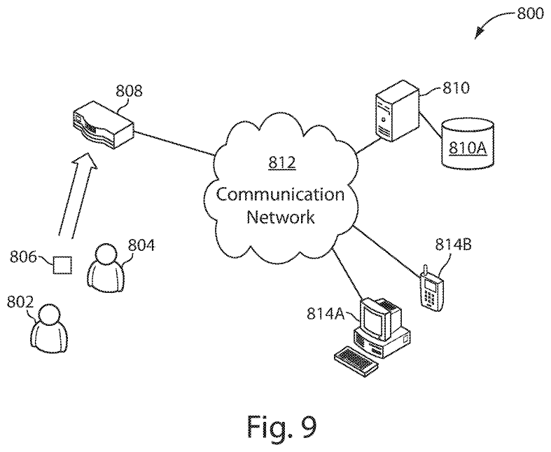

In some embodiments, the diagnostic techniques described above may be implemented via one or more computing devices executing one or more software facilities to analyze samples for a subject over time, measure nucleic acids (such as cell-free DNA) and/or noise, background or discordance QC in the samples, and produce a diagnostic result based on one or more of the samples. FIG. 9 illustrates an example of a computer system with which some embodiments may operate, though it should be appreciated that embodiments are not limited to operating with a system of the type illustrated in FIG. 9.

The computer system of FIG. 9 includes a subject 802 and a clinician 804 that may obtain a sample 806 from the subject 806. As should be appreciated from the foregoing, the sample 806 may be any suitable sample of biological material for the subject 802 that may be used to measure the presence of nucleic acids (such as cell-free DNA) and/or noise, background or discordance QC in the subject 802, including a blood sample. The sample 806 may be provided to an analysis device 808, which one of ordinary skill will appreciate from the foregoing will analyze the sample 808 so as to determine (including estimate) a total amount of nucleic acids (such as cell-free DNA) and/or an amount of a non-native nucleic acids (such as cell-free DNA) and/or noise, background or discordance QC in the sample 806 and/or the subject 802. For ease of illustration, the analysis device 808 is depicted as single device, but it should be appreciated that analysis device 808 may take any suitable form and may, in some embodiments, be implemented as multiple devices. To determine the amounts of nucleic acids (such as cell-free DNA) and/or noise, background or discordance QC in the sample 806 and/or subject 802, the analysis device 808 may perform any of the techniques described above, and is not limited to performing any particular analysis. The analysis device 808 may include one or more processors to execute an analysis facility implemented in software, which may drive the processor(s) to operate other hardware and receive the results of tasks performed by the other hardware to determine on overall result of the analysis, which may be the amounts of nucleic acids (such as cell-free DNA) and/or noise, background or discordance QC in the sample 806 and/or the subject 802. The analysis facility may be stored in one or more computer-readable storage media, such as a memory of the device 808. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

In some embodiments, the clinician 804 may directly provide the sample 806 to the analysis device 808 and may operate the device 808 in addition to obtaining the sample 806 from the subject 802, while in other embodiments the device 808 may be located geographically remote from the clinician 804 and subject 802 and the sample 806 may need to be shipped or otherwise transferred to a location of the analysis device 808. The sample 806 may in some embodiments be provided to the analysis device 808 together with (e.g., input via any suitable interface) an identifier for the sample 806 and/or the subject 802, for a date and/or time at which the sample 806 was obtained, or other information describing or identifying the sample 806.

The analysis device 808 may in some embodiments be configured to provide a result of the analysis performed on the sample 806 to a computing device 810, which may include a data store 810A that may be implemented as a database or other suitable data store. The computing device 810 may in some embodiments be implemented as one or more servers, including as one or more physical and/or virtual machines of a distributed computing platform such as a cloud service provider. In other embodiments, the device 810 may be implemented as a desktop or laptop personal computer, a smart mobile phone, a tablet computer, a special-purpose hardware device, or other computing device.

In some embodiments, the analysis device 808 may communicate the result of its analysis to the device 810 via one or more wired and/or wireless, local and/or wide-area computer communication networks, including the Internet. The result of the analysis may be communicated using any suitable protocol and may be communicated together with the information describing or identifying the sample 806, such as an identifier for the sample 806 and/or subject 802 or a date and/or time the sample 806 was obtained.

The computing device 810 may include one or more processors to execute a diagnostic facility implemented in software, which may drive the processor(s) to perform diagnostic techniques described herein. The diagnostic facility may be stored in one or more computer-readable storage media, such as a memory of the device 810. In other embodiments, techniques described herein for analyzing a sample may be partially or entirely implemented in one or more special-purpose computer components such as Application Specific Integrated Circuits (ASICs), or through any other suitable form of computer component that may take the place of a software implementation.

The diagnostic facility may receive the result of the analysis and the information describing or identifying the sample 806 and may store that information in the data store 810A. The information may be stored in the data store 810A in association with other information for the subject 802, such as in a case that information regarding prior samples for the subject 802 was previously received and stored by the diagnostic facility. The information regarding multiple samples may be associated using a common identifier, such as an identifier for the subject 802. In some cases, the data store 810A may include information for multiple different subjects.

The diagnostic facility may also be operated to analyze results of the analysis of one or more samples 806 for a particular subject 802, identified by user input, so as to determine a diagnosis for the subject 802. The diagnosis may be a conclusion of a risk that the subject 802 has, may have, or may in the future develop a particular condition. The diagnostic facility may determine the diagnosis using any of the various examples described above, including by comparing the amounts of nucleic acids (such as cell-free DNA) and/or noise, background or discordance QC determined for a particular sample 806 to one or more thresholds or by comparing a change over time in the amounts of nucleic acids (such as cell-free DNA) and/or noise, background or discordance QC determined for samples 806 over time to one or more thresholds. For example, the diagnostic facility may determine a risk to the subject 802 of a condition by comparing an amount of nucleic acids (such as non-native cell-free DNA) and/or noise, background or discordance QC for one or more samples 806 to a threshold. Based on the comparisons to the threshold(s), the diagnostic facility may produce an output indicative of a risk to the subject 802 of a condition.

As should be appreciated from the foregoing, in some embodiments, the diagnostic facility may be configured with different thresholds to which amounts of nucleic acids (such as cell-free DNA) and/or noise, background or discordance QC may be compared. The different thresholds may, for example, correspond to different demographic groups (age, gender, race, economic class, presence or absence of a particular procedure/condition/other in medical history, or other demographic categories), different conditions, and/or other parameters or combinations of parameters. In such embodiments, the diagnostic facility may be configured to select thresholds against which amounts of nucleic acids (such as cell-free DNA) and/or noise, background or discordance QC are to be compared, with different thresholds stored in memory of the computing device 810. The selection may thus be based on demographic information for the subject 802 in embodiments in which thresholds differ based on demographic group, and in these cases demographic information for the subject 802 may be provided to the diagnostic facility or retrieved (from another computing device, or a data store that may be the same or different from the data store 810A, or from any other suitable source) by the diagnostic facility using an identifier for the subject 802. The selection may additionally or alternatively be based on the condition for which a risk is to be determined, and the diagnostic facility may prior to determining the risk receive as input a condition and use the condition to select the thresholds on which to base the determination of risk. It should be appreciated that the diagnostic facility is not limited to selecting thresholds in any particular manner, in embodiments in which multiple thresholds are supported.

In some embodiments, the diagnostic facility may be configured to output for presentation to a user a user interface that includes a diagnosis of a risk and/or a basis for the diagnosis for a subject 802. The basis for the diagnosis may include, for example, amounts of nucleic acids (such as cell-free DNA) and/or noise, background or discordance QC detected in one or more samples 806 for a subject 802. In some embodiments, user interfaces may include any of the examples of results, values, amounts, graphs, etc. discussed above. They can include results, values, amounts, etc. over time. For example, in some embodiments, a user interface may incorporate a graph similar to that shown in any one of the figures provided herein. In such a case, in some cases the graph may be annotated to indicate to a user how different regions of the graph may correspond to different diagnoses that may be produced from an analysis of data displayed in the graph. For example, thresholds against which the graphed data may be compared to determine the analysis may be imposed on the graph(s).

A user interface including a graph, particularly with the lines and/or shading, may provide a user with a far more intuitive and faster-to-review interface to determine a risk of the subject 802 based on amounts of nucleic acids (such as cell-free DNA) and/or noise, background or discordance QC, than may be provided through other user interfaces. It should be appreciated, however, that embodiments are not limited to being implemented with any particular user interface.

In some embodiments, the diagnostic facility may output the diagnosis or a user interface to one or more other computing devices 814 (including devices 814A, 814B) that may be operated by the subject 802 and/or a clinician, which may be the clinician 804 or another clinician. The diagnostic facility may transmit the diagnosis and/or user interface to the device 814 via the network(s) 812.

Techniques operating according to the principles described herein may be implemented in any suitable manner. Included in the discussion above are a series of flow charts showing the steps and acts of various processes that determine a risk of a condition based on an analysis of amounts of nucleic acids (such as cell-free DNA) and/or noise, background or discordance QC. The processing and decision blocks discussed above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that embodiments are not limited to any particular syntax or operation of any particular circuit or of any particular programming language or type of programming language. Rather, one skilled in the art may use the description above to fabricate circuits or to implement computer software algorithms to perform the processing of a particular apparatus carrying out the types of techniques described herein. It should also be appreciated that, unless otherwise indicated herein, the particular sequence of steps and/or acts described above is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as a portion of a computing device or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 9, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Any one of the aforementioned, including the aforementioned devices, systems, embodiments, methods, techniques, algorithms, media, hardware, software, interfaces, processors, displays, networks, inputs, outputs or any combination thereof are provided herein in other aspects.

1. A method of assessing an amount of non-native nucleic acids in a sample from a subject at risk of, having or suspected of having cancer and/or noise, background or discordance QC, the sample comprising non-native and native nucleic acids, the method comprising:
1) for a plurality of single nucleotide variant (SNV) targets, performing an amplification-based quantification assay on the sample, or portion thereof, with at least two primer pairs, wherein each primer pair comprises a forward primer and a reverse primer, wherein one of the at least two primer pairs comprises a 3′ penultimate mismatch in a primer relative to one allele of the SNV target but a 3′ double mismatch relative to another allele of the SNV target and specifically amplifies the one allele of the SNV target, and the another of the at least two primer pairs specifically amplifies the another allele of the SNV target, and wherein at least one of the SNV targets is not a cancer-specific SNV target, or
2) for a plurality of SNV targets performing a sequencing-based assay on the sample, or portion thereof, and wherein at least one of the SNV targets is not a cancer-specific SNV target,
and obtaining or providing results from the amplification-based quantification or sequencing-based assay to determine the amount of non-native nucleic acids and/or the background, noise or discordance QC in the sample.

2. The method of claim 1, wherein none of the SNV targets are cancer-specific SNV targets.

3. (canceled)

4. The method of claim 1, wherein the method further comprises determining the amount of the non-native nucleic acids in the sample and/or noise, background or discordance QC based on the results.

5. The method of claim 1, wherein the results comprise the amount of the non-native nucleic acids and/or noise, background or discordance QC in the sample.

6. The method of claim 1, wherein the method further comprises determining the level of background, noise or discordance QC based on the results.

7. The method of claim 6, wherein the level of background, noise, or discordance QC is determined by performing the amplification-based quantification or sequencing-based assay using at least one genomic target that is homozygous and on the same allele in both the non-native and native nucleic acids, wherein the resulting minor species is indicative of the level of background, noise, or discordance QC.

8. A method of assessing an amount of non-native nucleic acids in a sample from a subject at risk of, having or suspected of having cancer and/or noise, background or discordance QC, the sample comprising non-native and native nucleic acids, the method comprising:
1) obtaining results from an amplification-based quantification assay performed on the sample, or portion thereof, wherein the assay comprises amplification of a plurality of single nucleotide variant (SNV) targets with at least two primer pairs for each of the SNV targets, wherein each primer pair comprises a forward primer and a reverse primer, wherein one of the at least two primer pairs comprises a 3′ penultimate mismatch in a primer relative to one allele of the SNV target but a 3′ double mismatch relative to another allele of the SNV target and specifically amplifies the one allele of the SNV target, and another of the at least two primer pairs specifically amplifies the another allele of the SNV target, and wherein at least one of the SNV targets is not a cancer-specific SNV informative target, or
2) obtaining results from a sequencing-based assay performed on the sample, or portion thereof, wherein the assay comprises sequencing of a plurality of SNV targets, and wherein at least one of the SNV targets is not a cancer-specific SNV informative target,
and assessing the amount of non-native nucleic acids and/or noise, background or discordance QC based on the results.

9. The method of claim 8, wherein the amount of the non-native nucleic acids in the sample and/or noise, background or discordance QC is based on the results of the amplification-based quantification or sequencing-based assays.

10. (canceled)

11. The method of claim 1, wherein the another primer pair of the at least two primer pairs also comprises a 3′ penultimate mismatch relative to the another allele of the SNV target but a 3′ double mismatch relative to the one allele of the SNV target in a primer and specifically amplifies the another allele of the SNV target.

12-16. (canceled)

17. The method of claim 1, wherein the amount of non-native nucleic acids in the sample is at least 5%.

18-20. (canceled)

21. The method of claim 1, wherein the amount of noise, background or discordance QC is at least 0.1%.

22-26. (canceled)

27. The method of claim 1, wherein the sample comprises cell-free DNA and the amount is an amount of non-native cell-free DNA and/or noise, background or discordance QC related to the quantification thereof.

28. (canceled)

29. The method of claim 1, wherein the subject has or is at risk of having a hematological cancer.

30. (canceled)

31. The method of claim 1, wherein the amplification-based quantification assays are quantitative PCR assays, such as real time PCR assays or digital PCR assays.

32. The method of claim 1, wherein the method further comprises determining a risk associated with cancer in the subject based on the amount of non-native nucleic acids and/or noise, background or discordance QC in the sample.

33-34. (canceled)

35. The method of claim 1, wherein the method further comprises selecting a treatment for the subject based on the amount of non-native nucleic acids and/or noise, background or discordance QC in the sample.

36. (canceled)

37. The method of claim 1, wherein the method further comprises treating the subject based on the amount of non-native nucleic acids and/or noise, background or discordance QC in the sample.

38. (canceled)

39. The method of claim 1, wherein the method further comprises monitoring or suggesting the monitoring of the amount of non-native nucleic acids and/or noise, background or discordance QC in the subject over time.

40. (canceled)

41. The method of claim 1, wherein the method further comprises evaluating an effect of a treatment administered to the subject based on the amount of non-native nucleic acids and/or noise, background or discordance QC.

42-45. (canceled)

46. A method comprising:
obtaining the amount of non-native nucleic acids and/or noise, background or discordance QC based on the method of claim 1, and
assessing a risk associated with cancer in a subject based on the levels or amount.

47-52. (canceled)
